(19) United States
(12) Patent Application Publication
(10) Pub. No.: US 2015/0030542 A1
(43) Pub. Date: Jan. 29, 2015

(54) METHODS FOR MEDICAL IMAGING

(71) Applicant: Sunil Singhal, Moorestown, NJ (US)

(72) Inventor: Sunil Singhal, Moorestown, NJ (US)

(21) Appl. No.: 13/951,841

(22) Filed: Jul. 26, 2013

(51) Int. Cl. A61K 49/00; A61N 5/10; A61B 5/00

(52) U.S. Cl. CPC A61K 49/0034; A61B 5/0071; A61N 5/1077
USPC 424/9.6; 600/476; 600/1

(57) ABSTRACT

The invention relates to methods of identifying, detecting, and locating a tissue(s), nodule(s) or mass(es) and its draining lymph nodes that is/are suspected to be abnormal, typically a neoplasm (i.e., cancer, malignancy, premalignancy) in an individual undergoing an invasive procedure (i.e., surgery or endoscopy) or a non-invasive procedure (ie. radiology). The method involves the use of, for example, indocyanine green (ICG). The uptake of this dye is different by diseased tissues and lymph nodes compared to non-diseased tissues when administered at the appropriate combination of dose and time and monitored with an appropriate device that can excite and capture the signal.

### BACKGROUND OF THE INVENTION

1. Field of Invention

The invention relates to methods of identifying, detecting, and locating a tissue(s), nodule(s) or mass(es) and its draining lymph nodes that is/are suspected to be abnormal, typically a neoplasm (i.e., cancer, malignancy, premalignancy) in an individual undergoing an invasive procedure (i.e., surgery or endoscopy) or a non-invasive procedure (ie. radiology). The method involves the use of, for example, indocyanine green (ICG). The uptake of this dye is different by diseased tissues and lymph nodes compared to non-diseased tissues when administered at the appropriate combination of dose and time and monitored with an appropriate device that can excite and capture the signal.

2. Description of Related Art

The invention relates to methods of identifying, detecting, and locating a tissue, nodule or mass that is suspected to be abnormal, typically a neoplasm (i.e., cancer, malignancy, premalignancy) in an individual undergoing surgery. The method involves the use of, for example, indocyanine green (ICG). The uptake of this dye is different by diseased tissues compared to non-diseased tissues when administered at the appropriate combination of dose and time with an appropriate device that can excite and capture the signal. Unlike prior technologies, this technique uses a systemic delivery of ICG to identify diseased tissues and draining lymph nodes.

Multiple technologies currently exist which allow tumor cells to fluoresce in animal models. Fluorescent proteins, such as green fluorescent protein (GFP), quantum dots and organic dyes can be used to tag and visualize cancer cells and specific cancer processes such as tumor growth, cell motility, invasion, and angiogenesis. (Hoffman 2005) These approaches are highly useful to study the biology of tumors, but, they have had limited success in humans due to the lack of tumor access, toxicity, dearth of clinical approved probes and paucity of large scale imaging devices. Furthermore, current approaches in humans require direct intratumoral injection of a fluorophore which necessitates a priori knowledge of all tumor deposits. (Schaafsma 2011, Schulz 2010, Choi 2010) A recent approach in humans utilized a folate-fluorescein fluorophore preparation specific to ovarian tumors but was limited by false negatives in folate-receptor negative tumors and the use of fluorescein which can be difficult to differentiate from autofluorescence. (van Dam 2011). The inventors have found that all solid human tumors could be fluorescently labeled by systemic injection of a fluorophore, which will have enormous scientific and clinical impact.

Nanoparticles are thought to accumulate in solid tumors due to a phenomenon known as the enhanced permeability and retention (EPR) effect. (Singhal 2010). The EPR effect, first described in 1986 by Matsumura and Maeda, is a property by which molecules such as nanoparticles passively collect in tissues due to the presence of defective endothelial cells and wide fenestrations (600 to 800 nm) in newly forming blood vessels. (Matsumura 1986). Neovasculature in cancer tissues and inflammatory lesions have higher pressure concentrations and lack the ability to respond to vasoactive mediators further promoting accumulation of nanoparticles. Once in the tumor microenvironment, nanoparticles are retained due to global properties such as molecular size, shape, charge and polarity, rather than tumor-specific targeting mechanisms such as ligand-receptor interactions. (Heneweer 2011). Although the EPR effect has shortcomings for delivering toxic payloads such as non-specific binding, we hypothesized that this property is well-suited for intraoperative removal of tumor masses, where specificity in less of a concern than sensitivity.

Near infrared (NW) contrast agents are the ideal imaging dyes for humans because the fluorescence can be detected at depths of 10 mm into the tissue. The excitation energy necessary for exciting MR contrast agents is low (10-1 eV) making it safe for use in humans without shielding. Indocyanine green (ICG) is a well-tolerated, non-toxic, inexpensive MR contrast agent that has been in clinical use for decades. (Henschen 1993, Donald 1973). It is the only NW contrast agent FDA approved for human use. ICG is a water-soluble, anionic, amphiphilic tricarbocyanine probe with a hydrodynamic diameter of 1.2 nm, and excitation and emission wavelengths in serum at 778 nm and 830 nm, respectively. (Polom 2011). Upon injection into the blood, 95% of ICG quickly binds to serum proteins (albumin, lipoproteins), and the resulting ICG-protein complex is 4-6 nm in size. (Yoneya 1998). As a consequence, the ICG-protein complex is delivered to most cancers and inflammatory tissues, thus it has the advantage of exquisite sensitivity for any abnormality.

Although several studies exist using ICG for imaging in humans, these approaches have used direct intratumoral injection and have not capitalized on the EPR properties of ICG. (Schaafsma 2011, Schulz 2010, Choi 2010). Our group and others have recently shown that the EPR effect may be feasible for delivering ICG to tumors of various histological subtypes in murine models. (Madajewski 2012, Kosaka 2011, Ishizawa 2009). The standard dose for intravenous dosing in humans is 0.2 to 0.4 mg/kg. However, for purposes of tumor targeting, the inventors have found a significantly higher dose to be necessary once the initial vascular clearance had occurred. This dose is above the FDA package labeling for ICG.

As a clinical application of this technology in humans, that the invention provides systemic delivery of a fluorescent contrast agent could dramatically improve intraoperative decisions during cancer surgery by identifying tumors by fluorescence. (Singhal 2010, Madajewski 2012). Surgery is the most effective therapy for solid tumors, and 700,000 cancer patients undergo resection for curative intent each year. (Aliperti 2011). However, despite a “curative” resection, up to 20% of patients develop local recurrences and the majority die within 2 years. (Aliperti 2011). Local recurrences occur due to retained tumor cells that are not recognized at the time of surgery and then quickly re-populate. (Predina 2013). The inventors realized that if human tumor tissue could fluoresce, more cancerous tissues would be identified during surgery resulting in superior disease clearance and potential reduction of local recurrences. Although this approach has exquisite sensitivity at the expense of specificity, the most important goal during surgery is to detect any abnormal tissue regardless of its origins as inflammatory or malignant. Thus, the fluorescent labeling of human tumors has enormous value during surgery. While the inventors examined the EPR effect in 11 human tumor types in this study, the results are broadly applicable to all solid tumors.

Fluorophores have revolutionized the study of tumor biology in vitro and in animal models, however, these technologies have had limited application to humans in vivo. To our knowledge, this is the first demonstration of labeling human cancer cells in vivo by systemic injection of a fluorescent near-infrared contrast agent. In fact, according to the knowledge in the field, ICG has limited potential and its application for tumor imaging will only be for sentinel lymph node when injected directly in the tumor. (See B. E. Schaafsma, J. S. Mieog, M. Hutteman, J. R. van der Vorst, P. J. Kuppen, C. W. Lowik, J. V. Frangioni, C. J. van de Velde, A. L. Vahrmeijer. The clinical use of indocyanine green as a near-infrared fluorescent contrast agent for image guided oncologic surgery, J Surg Oncol, 104 (2011) 323-332). This is an indication that the invention described herein is a new advance in the field.

The inventors show herein that nanoparticle-sized fluorescent agents do accumulate in solid tumors due to molecular properties rather than receptor-specific targeting. As a practical application, the inventors conducted a pilot study (n=27 patients) to determine if fluorescent labeling of 11 different types of tumors would identify cancer deposits during surgery (see Examples, hereinbelow). Despite preoperative imaging and a standard-of-care operation, surgeons were able to recognize extra tumor deposits in 2 patients (8%) by tumor fluorescence. In a third patient, tumor fluorescence from a resected breast lumpectomy specimen identified a close margin (<1 mm) that required immediate re-resection. These patients had a marked change in their clinical management.

All references cited herein are incorporated herein by reference in their entireties.

### BRIEF SUMMARY OF THE INVENTION

The invention provides a method for identifying abnormal tissue in a subject during an operative, radiologic or endoscopic procedure, said method comprising: (a) administering to the subject a fluorophore preparation comprising an effective amount of at least one fluorophore, wherein said at least one fluorophore comprises indocyanine green (ICG) in a total systemic dose of at least about 2 mg/kg of body weight of the subject, wherein the administration is systemic; (b) conducting said procedure after a waiting period subsequent to said administration, wherein said waiting period is selected from the group consisting of at least about 12 hours, about 24 hours, about 36 hours, about 48 hours, between about 12 to about 24 hours, between about 24 to about 36 hours, between about 36 to about 48 hours; (c) during the procedure, illuminating the area of interest with an illumination source emitting electromagnetic radiation (emr) having at least one wavelength which interacts with ICG dye, the emr having a wavelength of from about 600 nm to about 1000 nm; (d) imaging the abnormal tissue with an imaging device, wherein the abnormal tissue displays significantly more fluorescence caused by the fluorophore preparation; (e) optionally imaging the lymph nodes draining from the abnormal tissue; (f) optionally, treating sites of abnormal tissue by external beam radiation, laser therapy, or surgical removal. The invention further provides a method wherein said procedure is an operative procedure, radiologic or an endoscopic procedure. The invention further provides a method wherein said procedure is an endoscopic procedure. The invention further provides a method wherein the preparation is administered intravenously. The invention further provides a method wherein the fluorophore preparation comprises ICG administered in a total systemic dose of about 2 to 10 mg/kg of body weight of the subject. The invention further provides a method wherein the fluorophore preparation comprises ICG administered in a total systemic dose of at least about 2 to about 3 mg/kg of body weight of the subject.

The invention further provides a method wherein the fluorophore preparation further comprises a fluorophore selected from the group consisting of cyanine dyes, streptocyanines dyes, hemicyanine dyes, closed chain cyanine dyes, methylene blue (MB), IR-786, CW800-CA, and combinations thereof. The invention further provides a method wherein the abnormal tissue is selected from the group consisting of a neoplasia, a tumor, a metastasis, a lymph node, a sentinel lymph node, draining lymph node and combinations thereof. The invention further provides a method wherein the abnormal tissue is a neoplasia selected from the group consisting of breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal gangllioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, lymphomas, malignant melanomas, epidermoid carcinomas, lymph node, sentinel lymph node, and combinations thereof. The invention further provides a method wherein the abnormal tissue is pancreatic cancer, breast cancer, or colon cancer.

The invention further provides a method wherein said procedure further comprises treating sites of abnormal tissue by external beam radiation, laser therapy, and/or surgical removal. The invention further provides a method wherein said illumination source is selected from the group consisting of electron-stimulated, incandescent, halogen, electroluminescent, LED, gas discharge, xenon, laser, and laser diode. The invention further provides a method wherein said illumination source emits emr having at least one wavelength which interacts with ICG dye, the emr having a wavelength of at least 650 nm. The invention further provides a method wherein said illumination source emits emr having at least one wavelength which interacts with ICG dye, the emr having a wavelength of about 780 nm. The invention further provides a method wherein said imaging device is selected from the group consisting of spectrometer, digital, video camera, and CCD. The invention further provides a method wherein a combination of lights and filters is used to create the impression of a glowing abnormal tissue. The invention further provides a method further comprising imaging devices capable of capturing spectroscopic data from the tissue being imaged.

The invention further provides a method further comprising imaging devices to convert the near-infrared signal to a visible signal. The invention further provides a method wherein the imaging device is selected from the group consisting of devices which can be mounted over the patient, hand-held devices, devices which are attached to a long lens system, minimally invasive cameras, telescopes, endoscopes, esophagoscopes, colonoscopes, laparoscopes, thoracoscope long lens, capsule endoscopes, and combinations thereof. The invention further provides a method wherein the imaging device is ingested or implanted in the subject. The invention further provides a method wherein the imaging device can record scatter information from the signal that is being emitted from the excited fluorophore preparation in the abnormal tissue in order to improve the depth of penetration and imaging quality. The invention further provides a method wherein the imaging device comprises an optical coherence tomography device. The invention further provides a method wherein the imaging device is modified to excite different fluorophores separately and simultaneously capture the emission from the different fluorophores, further wherein computer software then represents this data simultaneously for an observer.

The invention provides a kit comprising a vial containing a sterile preparation of a fluorophore preparation for systemic administration comprising an effective amount of at least one fluorophore, wherein said at least one fluorophore comprises indocyanin green (ICG), and instructions for use, wherein said instructions direct administration of ICG at a total systemic dose of at least about 2 to 5 mg/kg of body weight of the subject, but up to 10 mg/kg, and direct a waiting period after administration of the fluorophore preparation is selected from the group consisting of about 12 hours, about 24 hours, about 36 hours, about 48 hours, between about 12 to about 24 hours, between about 24 to about 36 hours, between about 36 to about 48 hours. The invention further provides a kit wherein the fluorophore preparation further comprises a fluorophore selected from the group consisting of methylene blue (MB), IR-786, CW800-CA, and combinations thereof.

The invention provides a method for identifying abnormal tissue in a subject during an operative or endoscopic procedure, said method comprising: (a) administering to the subject a fluorophore preparation comprising an effective amount of at least one fluorophore, wherein said at least one fluorophore comprises indocyanin green (ICG), wherein the administration is systemic, further wherein the ICG is administered in a total systemic dose of about 2 to 10 mg/kg of body weight of the subject; (b) conducting said procedure after a waiting period subsequent to said administration, wherein said waiting period is at least about 12 hours; (c) during the procedure, illuminating the area of interest with an illumination source emitting electromagnetic radiation (emr) having at least one wavelength which interacts with ICG dye, the emr having a wavelength of from about 600 nm to about 1000 nm; (d) imaging the abnormal tissue, optionally with an imaging device, wherein the abnormal tissue displays significantly more fluorescence caused by the fluorophore preparation; (e) optionally imaging the lymph nodes draining from the abnormal tissue; (f) optionally, treating sites of abnormal tissue by external beam radiation, laser therapy, or surgical removal.

The invention provides a method for identifying abnormal tissue in a subject during an operative or endoscopic procedure, said method comprising: (a) administering to the subject a fluorophore preparation comprising an effective amount of at least one fluorophore, wherein said at least one fluorophore comprises indocyanine green (ICG), wherein the administration is systemic, further wherein the ICG is administered in a total systemic dose of at least about 2 to about 5 mg/kg of body weight of the subject, but up to 10 mg/kg; (b) conducting said procedure after a waiting period subsequent to said administration, wherein said waiting period is at least about 24 hours; (c) during the procedure, illuminating the area of interest with an illumination source emitting electromagnetic radiation (emr) having at least one wavelength which interacts with ICG dye, the emr having a wavelength of from about 600 nm to about 1000 nm; (d) imaging the abnormal tissue, optionally with an imaging device, wherein the abnormal tissue displays significantly more fluorescence caused by the fluorophore preparation; (e) optionally imaging the lymph nodes draining from the abnormal tissue; (f) optionally, treating sites of abnormal tissue by external beam radiation, laser therapy, or surgical removal.

### BRIEF DESCRIPTION OF SEVERAL VIEWS OF THE DRAWINGS

The invention will be described in conjunction with the following drawings. The patent or application file contains at least one drawing executed in color. Copies of this patent or patent application publication with color drawing(s) will be provided by the Office upon request and payment of the necessary fee.

FIG. 1. Preclinical evidence for NM tumor labeling to detect primary and metastatic tumor deposits. (A) Six cancer cell types were injected into the flank of syngeneic mice. Once established (200 mm3), animals were dosed with 7.5 mg/kg of ICG and imaged. Tumors were harvested, imaged and stained for CD31. Histology images taken at 200× magnification (B) C57bl/6 mice (n=21) were injected with LLC cells in their flanks on Day 0. Starting on Day 12, the animals were euthanized, dosed with 7.5 mg/kg ICG 20 hours earlier and their thoracic cavities opened. Observers determined if the metastatic tumor nodules were visible in the lung. NIR imaging was then used to detect disease that was not visible to the un-assisted human eye. Histology images taken at 100× (Mohs 2010).

FIG. 2. ICG can be delivered to human tumors by systemic delivery. (A) Tumor fluorescence in three representative histological tumor subtypes: lung cancer, thymic neoplasm and a carcinoid tumor. Standard CAT and PET imaging demonstrated the tumor location before surgery. Visual inspection alone cannot always discriminate the borders of tumor and normal tissue within an organ. Tumor fluorescence demonstrates tumor boundaries and differentiates normal tissue from diseased tissue. (B) Both in vivo and ex vivo imaging were used to quantitate fluorescence from tumors and normal tissue. Each specimen was measured at least 4 times. Tumor fluorescence was based on the mean of 5 different locations in the specimen.

FIG. 3. Tumor fluorescence was not correlated to (A) microvascular density and (B) tumor cell content. (C) Tumor ICG concentration was quantitated by simultaneous imaging of a standard panel of ICG alongside the tumor (Supplemental FIG. 1e). Images were imported into ImageJ®. Region of interst (ROI) data was taken from each of the 9 wells and from the tumor to quantitate the [ICG]. In addition, tumor biopsies were homogenized in some cases and placed in a hand held fluorometer. However, the signal was attenuated in situations that the homogenate was opaque, therefore, this approach may have been subject to technical error. Attempts at digestion disrupted the fluorescent signal. (D) Two representative tumors are shown by immunohistochemistry, NIR fluorescent microscopy and overlay images. Due to collateral signal, fine discrimination of the location of the ICG is not precise, however, suggests distribution in the tumor interstitium and bound to the cell surfaces.

FIG. 4. Identification of metastatic tumor deposits in Patient #02. (A) After opening the chest, visual and manual inspection of the right upper lobe (RUL) immediately identified the tumor (1^{st} upper panel). Strong fluorescence was seen in situ (2^{nd} upper panel). The presence of highly fluorescent tumor was confirmed when the lobe was examined ex vivo (3^{rd} and 4^{th} upper panel). The specimen was divided in half, and the interior of the tumor was also brightly fluorescent (5^{th} and 6^{th} upper panel). (B) After completing the right upper lobectomy, the right lower pulmonary lobe (RLL) did not appear to have any metastatic nodules on visual inspection (1^{st} lower panel). However, when examined using fluorescence in situ, suspicious areas were identified (white arrow, 2^{nd} lower panel). A 6 cm biopsy was excised from the RLL (3^{rd} lower panel) and the presence of highly fluorescent areas were confirmed ex vivo (white arrows, 4^{th} and 5^{th} lower panels). A rapid frozen section confirmed microscopic metastatic adenocarcinoma (6^{th} lower panel).

FIG. 5. Identification of a close margin (<1 mm) on a breast cancer lumpectomy. (A) Preoperative MRI demonstrated a breast nodule close to the pectoralis muscle (white arrow, 1^{st} upper panel). Intraoperatively, a standard lumpectomy was performed (2^{nd} upper panel). The tumor was fluorescing up to the resections margins in vivo (black arrow, 3^{rd} upper panel). (B) Ex vivo, the specimen did not appear to have residual tumor cells at the margin (1^{st} lower panel), however, tumor fluorescence suggested a close margin (2^{nd} lower panel). Final pathology ultimately confirmed <1 mm tumor margin from the initial specimen (3^{rd} lower panel).

FIG. 6. Clinical characteristics and fluorescent information from 27 patients who underwent surgery.

FIG. 7. Configuration of the intraoperative camera. (A) The operating room is configured with the patient lying on the table. The surgeon is situated to the right of the patient. The assistant surgeon is located to the left of the patient. The camera is hung above the patient from a secure beam. (B) Intraoperative photograph of the configuration of the operating room. (C) Intraoperative photograph of the surgeon's view of the patient and the display from the camera. (D) Schematic and photograph of the intraoperative camera. (E) Standard panel that is used to quantitate tumor fluorescence during ex vivo analysis.

### DETAILED DESCRIPTION OF THE INVENTION

The inventors show that intra-operative imaging with a near-infrared fluorescent dye will improve tumor detection, draining lymph node identification and/or resection. Optical techniques provide unique advantages which are not available with other imaging modalities. First, they do not require significant radiation. Thus, this technology is safe for patients as well as the personnel performing the procedure, making it more readily acceptable in the operating room. Second, although optical imaging has limited penetration depths due to tissue scattering and blood absorption, the lesions are surgically exposed and can be brought in close proximity to the imaging device such that they become accessible to optical illumination and detection. Alternative particles do exist which permit deeper tissue penetration, but they would require higher excitation energy sources and may not receive wide spread approval by surgeons due to their risk of desiccating the tissues and potential harm to the surgical staff. Lastly, optical techniques are intuitive for surgeons and do not require complex imaging manipulations.

We acknowledge that ICG is non-specific in nature. It diffuses into any regions of vascular permeability; hence, both inflammatory and neoplastic areas are equally likely to be fluorescent. This fact, however, does not limit its clinical application. For example, in this series, the surgeon detected fluorescence in an aspergillus infection. This lesion still required resection for diagnosis. It is sufficiently sensitive to detect almost any solid tumor. Preliminary studies in our group have also demonstrated this technology works in several other tumor types.

One of the most important findings was the lack of correlation between fluorescent data and characteristics of the tumor. Despite examining several variables including biodistribution, tumor cell density, and vascular density, there was no characteristic that had an impact on clinical usefulness. The inventors found that intraoperative imaging is exquisitely sensitive and can image tumors with even modest fluorescence and is not dependent on heavy neovascularization. One of the long-standing concerns of using the EPR effect to deliver toxic nanoparticles has been the lack of uniform distribution of agents, especially in diverse tumor types.(Singh 2012). However, the data demonstrated that in diagnostic imaging at the macroscopic level, subtle differences in the density of the contrast agent in different regions of the tumor are not important. In fact, all but one of the tumors was fluorescent irrespective of all the factors we examined. It is possible that there is significant collateral fluorescence that explains the uniform appearance of the tumor fluorescence. On a practical level, this suggests the robustness of this approach and the potential clinical value.

In conclusion, the ability to fluorescently label tumors in humans may have enormous clinical impact. Biologically, the ability to identify abnormal tissues by the EPR effect provides the opportunity to study the tumor microenvironment in fresh human tissue before embedding in paraffin. Clinically, the value of this technology is to draw attention to tissues that would otherwise not have been examined. These data could affect the indications and approaches for patients with cancer. Cytoreductive surgery may become more valuable for many cancers which were previously thought incurable by resection such as ovarian cancer and malignant mesothelioma. In patients with prior surgery and/or radiation-induced injury, image guided surgery could identify cancer deposits in a hostile surgical field. Furthermore, for minimally invasive and robotic operations where the surgeon has no benefit of manual palpation, image guidance can improve identification of tumor deposits. Finally, surgeons may be able to provide superior decision making in the operating room to change the course of an operation.

According to the present invention, the term “living body” covers the living body of a human or a non-human animal and the organs and tissues thereof, unless otherwise specified.

The terms “organ” and “tissue” are not particularly limited. Examples of an “organ” include the lung, esophagus, breast, stomach, liver, gallbladder, bile duct, pancreas, colon, rectum, bladder, prostate gland, and uterus. Examples of “tissue” include tissue of any such organ.

Further, such “organ” or “tissue” may be not only an in vivo organ or tissue but also an in vitro organ or tissue.

### Fluorophores

The term “fluorophore” as used herein refers to a composition that is inherently fluorescent. Fluorophores may be substituted to alter the solubility, spectral properties or physical properties of the fluorophore. Numerous fluorophores are known to those skilled in the art and include, but are not limited to coumarin, acridine, furan, dansyl, cyanine, pyrene, naphthalene, benzofurans, quinolines, quinazolinones, indoles, benzazoles, borapolyazaindacenes, oxazine and xanthenes, with the latter including fluoresceins, rhodamines, rosamine and rhodols as well as other fluorophores described in RICHARD P. HAUGLAND, MOLECULAR PROBES HANDBOOK OF FLUORESCENT PROBES AND RESEARCH CHEMICALS (9^{th} edition, including the CD-ROM, September 2002). As used herein fluorophores of the present invention are compatible with in vivo imaging, optically excited in tissue, and generally have an excitation wavelength of about 580 nm to about 900 nm or longer.

A fluorescent dye or fluorophore of the present invention is any chemical moiety that exhibits an absorption maximum beyond 580 nm and that is optically excited and observable in tissue. Dyes of the present invention include, without limitation; a pyrene, an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a carbocyanine (including any corresponding compounds in U.S. Ser. Nos. 09/968,401; 09/969,853 and 11/150,596 and U.S. Pat. Nos. 6,403,807; 6,348,599; 5,486,616; 5,268,486; 5,569,587; 5,569,766; 5,627,027; 6,664,047; 6,048,982 AND 6,641,798), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343 and U.S. Ser. No. 09/922,333), an oxazine or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs. Where the dye is a xanthene, the dye is optionally a fluorescein, a rhodol (including any corresponding compounds disclosed in U.S. Pat. Nos. 5,227,487 and 5,442,045), a rosamine or a rhodamine (including any corresponding compounds in U.S. Pat. Nos. 5,798,276; 5,846,737; 5,847,162; 6,017,712; 6,025,505; 6,080,852; 6,716,979; 6,562,632). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171). Fluorinated xanthene dyes have been described previously as possessing particularly useful fluorescence properties (Int. Publ. No. WO 97/39064 and U.S. Pat. No. 6,162,931).

Preferred dyes of the invention include ICG, MB, xanthene, cyanine (streptocyanines, hemicyanines, and closed chain cyanines), and borapolyazaindacene dyes or dyes sold under the trade name BODIPY.

ICG is an FDA approved, water-soluble tricarbocyanine dye routinely used in clinical settings for measuring cardiac output, liver function, and retinal angiography and has been in use for over 50 years. The chemical formula is C₄₅H₄₇N₂O₆S₂Na and the compound has a molecular weight of 774.96 Da (CAS number 3599-32-4). It has a peak absorption in the near-infrared spectrum at 805 nm and maximal emission at 835 nm. ICG is rapidly and completely bound to plasma proteins (especially albumin) after intravenous injection in the blood. At that point the emission spectrum shifts dramatically and can be excited to the 735 nm absorbance-770 nm emission spectrum.

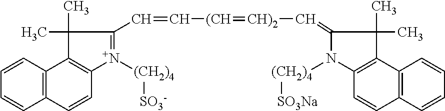

### Scott Prahl, Oregon Medical Laser Center

Indocyanine Green for Injection USP is a sterile, lyophilized green powder containing 25 mg of indocyanine green with no more than 5% sodium iodide. Indocyanine Green for Injection USP is dissolved using Sterile Water for Injection, and is to be administered intravenously. There is currently no known toxicity to this agent and no overdose has ever been reported.

### Mixing ICG With Other Fluorophores

An individual can receive multiple compounds that fluoresce (i.e., glow) before the operation. Different fluorophores are retained by different organs and structures. This allows the observer to discriminate and distinguish different tissues by the type of fluorophore. The imaging device can be modified to excite different fluorophores separately and simultaneously capture the emission from the different fluorophores. Computer software can then represent this data simultaneously for the observer. If this approach is taken, as long as ICG is part of the mixture of fluorophores, the ability of ICG to image a tumor is unchanged.

Although it was previously known to use methylene blue (MB) as a visual dye, the use of MB in fluorescence imaging has not been significantly appreciated. As described herein, methylene blue (MB) has fluorescent properties. The emission wavelength (670 nm to 720 nm with a peak that shifts as a function of dye concentration) is within the Near Infrared (NIR) range at physiologically safe concentrations and therefore permits high sensitivity and high signal to background due to low autofluorescence in humans and animals. This characteristic allows MB to be used as a vascular contrast agent, using fluorescence imaging technology. Surprisingly, MB is secreted or partitions specifically into certain fluids and organs, including the thoracic duct, bile (allowing visualization of biliary tree), urine (allowing visualization of the ureters), heart myocardium, vasculature (allowing imaging of, inter alia, the myocardium, cornonary artery, etc.), and pancreas (e.g., into beta cells, allowing visualization of that organ and tumors and metastases with a pancreatic origin, e.g., insulinomas).

MB has the advantage of already being approved by the U.S. Food & Drug Administration as a blue dye to assess gastrointestinal tube placement and as a treatment for methemoglobinemia. Doses of 1.0-2.0 mg/kg of methylene blue are widely used clinically for the treatment of methemoglobinaemia, and much larger doses (on the order of 4.0-7.5 mg/kg) are administered for parathyroidal adenoma/hyperplasia detection. At the higher end, e.g., 7.5 mg/kg, MB administration sometimes causes severe adverse reactions, e.g., methemoglobinaemia or anaphylaxis. In addition, there are some reports indicating that intradermal injection of MB can cause skin damage. For example, the high doses used for sentinel node detection, e.g., around 4 ml of 30 mM MB, are associated with reports of injection site reactions. At these high concentrations, no fluorescence would be visible due to the concentration-dependent quenching of MB emissions. In general, the total dose that will be used for most applications is about 1-4 mg/kg of body weight when administered systemically.

CW800-CA is a carboxylic acid analog of IRDye®800CW, a newer heptamethine indocyanine with higher quantum yields and molar extinction coefficients. IR-786 is a heptamethine indocyanine with no sulphonation, and is an extremely hydrophobic agent. On the other hand, CW800-CA is a tetra-sulphonated heptamethine indocyanine, which increases its hydrophilicity.

CW800-CA (LI-COR Inc.): The carboxylic acid of IRDye®800-CW prepared from the commercially available N-hydroxysuccinimide ester, by hydrolysis of the ester in water at pH 8.5. This is a tetra-sulphonated heptamethine indocyanine with emission.apprxeq.800 nm. After intravenous injection it is rapidly cleared by: 1) the liver and excreted into bile and 2) the kidneys and excreted into urine. Thus, this dye is useful for imaging the biliary tree and ureters.

IR-786 (Sigma-Aldrich, Inc.): Commercially available non-sulphonated near-infrared heptamethine indocyanine fluorophore. After intravenous injection, it is rapidly extracted into many tissues in the body, especially the liver, and is inefficiently transported into bile. IR-786 can be used to image the structures described herein.

IRDye78: Commercially available tetra-sulfonated heptamethine indocyanine-type NIR fluorophore with peak absorption at 772 nm and peak emission at 790 nm. IRDye78 can be used to image the structures described herein when administered by direct injection or cannulation of the structure. See, e.g., Zaheer et al., Mol. Imaging, 2002; 1(4):354-64.

### Dosage and Administration

An individual who has a suspected or unsuspected abnormal nodule or mass that warrants surgery can be systemically injected with, for example, indocyanine green at a dose of 2 to 10 mg/kg through a peripheral vein with minimal to no toxicity. Increasing the dose will increase the fluorescence until quenching occurs. The injection should not be done as a sudden bolus due to safety concerns for the individual. This method can be used to identify any solid abnormal tissue or cancer, and does not necessarily extend to liquid tumors (ie. lymphoma and leukemia). In order to improve the quality of the signal, the ICG should be kept away from excitation light sources in the preparation and administration of the dye.

After at least 12 hours, ideally 24 hours, but up to 48 hours, that patient can undergo surgery and the nodule in question can be imaged real-time and be found to be fluorescent. If this time is not waited, there will be an excessive amount of background noise which will not allow adequate discrimination of normal to abnormal tissue, nodule or mass. This fluorescence will exceed the background signal (i.e., noise) from the surrounding normal tissues that will allow one to select what tissue is abnormal and what tissue is normal (i.e., not diseased). After removing the abnormal tissue it will retain its fluorescence if kept in darkness without any excitation from a light source. In order to improve the quality of the signal that is captured, reduce the exposure time to excitation light sources in the near-infrared. However, photobleaching is rarely a problem.

### Administering the Fluorophore Preparation

Administration of a fluorophore preparation provided herein can be effected by any method that enables delivery of the fluorophore preparation to the site of the abnormal tissue, such as cancer or suspected cancer. In one embodiment, delivery is via circulation in the bloodstream. To place the fluorophore preparations in contact with cancerous tissues or cells, the methods of administration include oral, buccal intraduodenal, parenteral injection (including intravenous, subcutaneous, intramuscular, intravascular, or infusion), topical administration, and rectal.

The amount of the fluorophore preparation administered will be dependent upon the subject being treated, the severity of the cancer, localization of the cancer, the rate of administration, the disposition of the fluorophore preparation (e.g., solubility and fluorescence intensity) and the discretion of the administrator. However, an effective dosage is typically in the range of about 0.001 to about 100 mg per kg body weight, preferably about 1 to about 35 mg/kg/day, preferably about 2 to about 10 mg/kg/day, preferably about 2 to about 5 mg/kg/day, but up to 10 mg/kg in single or divided doses. In some instances, dosage levels below the lower limit of the aforesaid range may be more than adequate, while in other cases still larger doses may be employed without causing any harmful side effect, although such larger doses may be divided into several smaller doses for administration throughout the day.

The imaging fluorophore preparation may, for example, be in a form suitable for oral administration, such as a tablet, capsule, pill, powder, sustained release formulation, solution, or suspension; for parenteral injection, such as a sterile solution, suspension or emulsion; for topical administration, such as an ointment or cream; or for rectal administration, such as a suppository. The imaging fluorophore preparation may be in unit dosage forms suitable for single administration of precise dosages and can include a conventional pharmaceutical carrier or excipient.

Exemplary parenteral administration forms include solutions or suspensions of the imaging fluorophore preparation in sterile aqueous solutions, for example, aqueous propylene glycol or dextrose solutions. Such dosage forms can be suitably buffered, if desired.

Suitable pharmaceutical carriers include inert diluents or fillers, water, and various organic solvents. The pharmaceutical compositions may, if desired, contain additional ingredients such as flavorings, binders, excipients, and the like. Thus for oral administration, tablets containing various excipients, such as citric acid, may be employed together with various disintegrants such as starch, alginic acid, and certain complex silicates, and with binding agents such as sucrose, gelatin, and acacia. Additionally, lubricating agents such as magnesium stearate, sodium lauryl sulfate, and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the imaging fluorophore fluorophore preparation therein may be combined with various sweetening or flavoring agents, coloring matters or dyes, and, if desired, emulsifying agents or suspending agents, together with diluents such as water, ethanol, propylene glycol, glycerin, or combinations thereof.

Methods of preparing various pharmaceutical compositions with a specific amount of an active ingredient that are suitable for use with the active imaging fluorophore fluorophore preparations of the present invention are known, or will be apparent upon consideration of the disclosure herein, to those skilled in this art. For examples, see Remington's Pharmaceutical Sciences, Mack Publishing Company, Easter, Pa., 15th Edition (1975).

Because the imaging fluorescent preparation of the present invention are preferentially taken up by cancer cells, it is possible to obtain an image of or visually confirm the presence of cancer cells that have taken up the preparation. Detection of the preparations can be performed using essentially any fluorescence detection device to obtain an image of the cancerous tissues or cells.

A “diagnostically effective amount” means an amount of a compound that, when administered to a subject for screening for tumors, is sufficient to provide a detectable distinction between a benign structure and a neoplasia. The “diagnostically effective amount” will vary depending on the compound, the condition to be detected, the severity or the condition, the age and relative health of the subject, the route and form of administration, the judgment of the attending medical or veterinary practitioner, and other factors.

A fluorophore preparation of the present invention is administered to a subject in a diagnostically effective amount. A compound of the present invention can be administered alone or as part of a pharmaceutically acceptable composition. In addition, a compound or composition can be administered all at once, as for example, by a bolus injection, multiple times, such as by a series of tablets, or delivered substantially uniformly over a period of time, as for example, using transdermal delivery. It is also noted that the dose of the compound can be varied over time. A compound of the present invention can be administered using an immediate release formulation, a controlled release formulation, or combinations thereof. The term “controlled release” includes sustained release, delayed release, and combinations thereof. In preferred embodiments, a fluorescent compound of the present invention is combined with a pharmaceutically acceptable carrier to produce a pharmaceutical preparation for parenteral administration.

The term “pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

As defined herein, “contacting” means that the fluorescent compound used in the present invention is introduced to a sample containing cells or tissue in a test tube, flask, tissue culture, chip, array, plate, microplate, capillary, or the like, and incubated at a temperature and time sufficient to permit binding of the fluorescent compound to a receptor or intercalation into a membrane. Methods for contacting the samples with the fluorescent compound or other specific binding components are known to those skilled in the art and may be selected depending on the type of assay protocol to be run. Incubation methods are also standard and are known to those skilled in the art.

In another embodiment, the term “contacting” means that the fluorescent compound used in the present invention is introduced into a patient receiving treatment, and the compound is allowed to come in contact in vivo. In further embodiment, the term “contacting” means that the fluorescent compound used in the present invention is introduced into a patient requiring screening for tumors, and the compound is allowed to come in contact in vivo.

The invention also generally relates to compositions comprising the compounds of the present invention.

As used herein, the term “composition” is intended to encompass a product comprising the specified ingredients in the specified amounts, as well as any product which results, directly or indirectly, from a combination of the specified ingredients in the specified amounts.

In some embodiments, the pharmaceutical composition is administered parenterally, paracancerally, transmucosally, tansdermally, intramuscularly, intravenously, intradermally, subcutaneously, intraperitonealy, intraventricularly, intracranially and intratumorally.

Further, as used herein “pharmaceutically acceptable carriers” are well known to those skilled in the art and include, but are not limited to, 0.01-0.1 M and preferably 0.05M phosphate buffer or 0.9% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

The fluorophore preparations administrable by the invention can be prepared by known dissolving, mixing, granulating, or tablet-forming processes. For oral administration, the tumor-specific ether analogs or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intra-arterial, or intramuscular injection), the tumor-specific ether analogs or their physiologically tolerated derivatives such as salts, esters, N-oxides, and the like are converted into a solution, suspension, or expulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

The preparation of pharmaceutical compositions which contain an active component is well understood in the art. Such compositions may be prepared as aerosols delivered to the nasopharynx or as injectables, either as liquid solutions or suspensions; however, solid forms suitable for solution in, or suspension in, liquid prior to injection can also be prepared. The preparation can also be emulsified. Active therapeutic ingredients are often mixed with excipients which are pharmaceutically acceptable and compatible with the active ingredient. Suitable excipients are, for example, water, saline, dextrose, glycerol, ethanol, or the like or any combination thereof.

In addition, the composition can contain minor amounts of auxiliary substances such as wetting or emulsifying agents, pH buffering agents which enhance the effectiveness of the active ingredient.

### Methods of Use

The compounds of the present invention may be used in a variety of diagnostic and therapeutic methods.

In one embodiment, the compounds may administered to the patient via either the enteral, intravenous or parenteral routes (i.e., orally or via IV) for the surgical, endoscopic or radiographic determination of the presence of internal neoplasia. Examples include, but are not limited to, endoscopic diagnosis of malignancy in the colon, rectum, small bowel, esophagus, stomach, duodenum, uterus, pancreas and common bile duct, bronchi, esophagus, mouth, sinus, lung, bladder, kidney, abdominal cavity or thoracic (chest) cavity.

In a preferred embodiment, the invention provides a method for radiographically, surgically or endoscopically distinguishing a benign tissue from a malignant tissue in a selected region by using an endoscope or an open cavity system having at least two wavelength in a patient comprising the steps of: (a) administering a fluorescently labeled compound to the patient; (b) using a first technique to produce a visualization of the anatomy of the selected region using the first wavelength of a scope; (c) using a second technique to produce a visualization of the distribution of fluorescence produced by the fluorophore composition; and (d) comparing the visualization of the anatomy of the selected region by the first wavelength to the visualization of the distribution of fluorescence by the second wavelength produced by the fluorophore composition thereby distinguishing a benign tissue from malignant tissue.

In another embodiment, the compounds may be used to aid in the selection of biopsy tissues.

In another embodiment, the compounds may be used to aid in identification of abnormal tissue through the skin via optical coherence and other technology that captures scatter data from the fluorescent dye.

In yet another embodiment, the compounds may be administered to the patient via either the enteral or parenteral routes or via topical application for the visual and/or microscopically aided determination of the presence of malignant lesions on the skin. Examples include, but are not limited to, differentiating between benign and malignant lesions on the skin.

In another embodiment, the compounds may be used to aid in the selection of biopsy tissues in the above-listed skin malignancies.

In yet another embodiment, the compounds may be used to aid in the determination of malignant tissue margins during operative resection or Mohs surgery of such lesion.

In another embodiment, the compounds may be administered to the patient via either the enteral or parenteral routes (i.e. orally or IV) for the visual and or microscopic-aided determination of the presence of malignant tissue at the borders of known malignancies during surgery. Examples include, but are not limited to, the intraoperative determination of the borders of a malignancy to aid the complete biopsy and/or surgical resection of said malignancy. These methods can be used for any malignancy in any tissue of the human body.

In yet another embodiment, the compounds may be used to determine the presence of residual malignant cells in a pathological specimen that has been excised from the body of the patient and/or to determine the presence of residual cancer cells in situ in a patient.

For example, in one embodiment, the invention provides a method of determining the presence of residual malignant cells in a patient undergoing cancer therapy comprising (a) administering to a patient undergoing said cancer therapy the fluorophore composition; (b) visualizing the tissue that was determined to be malignant prior to said cancer therapy; and (c) assessing accumulation of the fluorophore composition in said tissue, wherein an accumulation of said fluorescent compound in said tissue indicates a possible presence of residual malignant cells.

In yet another embodiment, the invention provides a method of determining the presence of residual malignant cells in a patient undergoing cancer therapy comprising (a) excising a pathological specimen from a patient undergoing said cancer therapy; b) incubating said pathological specimen with the fluorophore composition; and (c) visualizing the distribution of said fluorophore composition in said pathological specimen; wherein an accumulation of said fluorophore composition in said specimen indicates a possible presence of residual malignant cells.

In another embodiment, the invention can provide a method for identifying the lymph nodes that drain from a diseased tissue. The fluorophore will accumulate then drain from the diseased tissue to the draining lymph node which can be identified. This is independent from a process where the fluorophore in directly injected into the diseased tissue. This is a systemic delivery of the fluorophore.

In yet another embodiment, the provided compounds may be used for tumor therapy response monitoring. In a preferred embodiment, the invention provides a method of monitoring response to a tumor therapy comprising (a) administering to a patient prior to said tumor therapy the fluorophore composition; (b) providing said tumor therapy; (c) providing the fluorophore composition after the tumor therapy; and (d) assessing difference in accumulation of the fluorophore composition from step (a) and step (c), wherein a greater accumulation of the compound in step (a) versus lesser accumulation in step (c) indicates a positive response to the treatment and/or an effective treatment methodology.

### Detection of Cancer

Detection and imaging of tissues or cells that take up the fluorophore preparations described herein can be accomplished using visual techniques or via two-dimensional image information processing by direct continuous observation with a fluorescence microscope or any capture device with fluorescent capabilities. While spatial resolution can be difficult for certain visual methods (unaided by spectral enhancers or microscopes), a typical fluorescence microscope can provide sufficient resolution at a single cell level.

For example, with a confocal laser scanning fluorescent microscope, 3-dimensional stereoscopic image information with a resolution of about 1 micron can be continuously obtained in real time from tissues in vivo. A variety of known methods can be adapted for use with the fluorophore preparations of the present invention. For example, the fluorophore preparations of the invention can be used in the endoscopic technique described in U.S. Pat. No. 5,261,410, in which an infrared monochromatic light source is employed and the Raman shift in emission radiation is measured to assess the tissue. Likewise, PCT patent publication No. WO 96/10363 discloses a method of normalization by dividing the intensity at each wavelength by the integrated area under the spectrum. Differences in the resulting curves are then used as the basis for diagnosis.

One of skill in the art will appreciate that essentially any fluorescence detection means, either microscopic or macroscopic, can be employed that is capable of detecting the fluorophore preparationlocalized in a particular lesion, tissue, organ, or cell.

In some embodiments, the detection means can be in the form of an endoscope inserted into a body cavity through an orifice, such as the mouth, nose, ear, anus, urethra, vagina or an incision. The term “endoscope” is used here to refer to any scope introduced into a body cavity, e.g., an anally introduced endoscope, an orally introduced bronchoscope, a urethrally introduced cystoscope, an abdominally introduced laparoscope, and the like. The miniaturization of scope components has greatly enhanced the utility of an endoscope, making endoscopes particularly useful in the practice of the present invention.

In addition to methods of detecting cancer as generally described above, certain embodiments of the present invention relate to intraoperative, laparoscopic, intravascular, and endoscopic examination, biopsy and treatment of tissues and/or organs with a fluorophore preparation detecting means capable of close approach to suspected sites of tumor recurrence, metastasis, or incomplete removal of cancer tissue. As used herein, endoscopic procedures include laparoscopic procedures.

Embodiments of the present invention also relate to the intravascular, intraoperative, laparoscopic, and endoscopic examination of lesions with a fluorophore preparation detecting means capable of close approach to suspected sites of the lesions, especially non-malignant pathological lesions. Lesions include cancerous, hyperplasic, and pre-cancerous cells or tissues.

In one embodiment, a surgeon or clinician, through the use of, e.g., an intraoperative, laparoscopic, intravascular probe or an endoscope, can quickly scan areas of suspected tumor growth and use the level of fluorescence to more precisely discriminate tumor tissue from non-tumor tissue and thereby more precisely define tumor borders for surgical resection or diagnostic evaluation, or for laser or radiation therapy, including brachytherapy and external beam therapy, or for improved biopsy procedures.

Other embodiments enable the intravascular, intraoperative, laparoscopic, or endoscopic detection means to be similarly used to define and treat lesions. In another embodiment, the fluorophore preparation is useful for therapy of the detected tumor by emitting oxygen free radicals or other byproducts which damage the cells in which there has been accumulation of the fluorophore preparation. The emission of such damaging agents can be aided or induced by the energy which excites the fluorophore.

The above detection methods can be carried out in combination with a surgical procedure, such as a cancer resection. The method of detecting can be carried out endoscopically, for example, or visually as part of a skin examination for melanoma screening.

During the procedure, and depending upon the fluorophore preparation used, detection can be visual. In some embodiments, the fluorescence of cells that have taken up the fluorophore preparation can be enhanced by excitation of the fluorophore with light of a suitable wavelength. Accordingly, once a portion of the tumor or lesion is removed, the remaining tissue can be subjected to a suitable light source to excite the fluorescent fluorophore preparations that remain and additional resection can be accomplished.

In other embodiments, detection can be accomplished using fluoroscopes and other detection devices known to those of skill in the art.

In another embodiment, the dye can be detected in abnormal tissues below the skin by a capture device that can locate the accumulated fluorophore through measuring the scatter of the signal from the fluorophore after any excitation technique.

### Methods of Detecting or Imaging Pre-Cancer

In a related aspect, the present invention provides methods for detecting pre-cancerous cells in a subject, comprising:

(a) administering to the subject an effective amount of a fluorophore composition; and

(b) detecting cells that take up the fluorophore preparation to determine if pre-cancerous cells are present in the subject.

### Abnormal Tissue

In accordance with one or more embodiments of the present invention, it will be understood that the types of abnormal tissue may include, for example, cancerous or pre-cancerous, as well as lymph nodes, including for example sentinel lymph nodes.

In accordance with one or more embodiments of the present invention, it will be understood that the types of abnormal tissue identification/diagnosis which may be made, using the methods provided herein, is not necessarily limited. For purposes herein, the abnormal tissue may be a neoplasia selected from the group consisting of breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronms, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, lymphomas, malignant melanomas, epidermoid carcinomas, lymph node, sentinel lymph node, and combinations thereof.

In accordance with one or more embodiments of the present invention, it will be understood that the types of cancer diagnosis which may be made, using the methods provided herein, is not necessarily limited. For purposes herein, the cancer can be any cancer. As used herein, the term “cancer” is meant any malignant growth or tumor caused by abnormal and uncontrolled cell division that may spread to other parts of the body through the lymphatic system or the blood stream. The cancer can be any cancer, including any of acute lymphocytic cancer, acute myeloid leukemia, alveolar rhabdomyosarcoma, adenocarcinoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor. Hodgkin lymphoma, hypopharynx cancer, hepatocellular cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer.

The cancer can be an epithelial cancer. As used herein the term “epithelial cancer” refers to an invasive malignant tumor derived from epithelial tissue that can metastasize to other areas of the body, e.g., a carcinoma. In a preferred embodiment, the epithelial cancer is breast cancer. Alternatively, the cancer can be a non-epithelial cancer, e.g., a sarcoma, leukemia, myeloma, lymphoma, neuroblastoma, glioma, or a cancer of muscle tissue or of the central nervous system (CNS).

The cancer can be a non-epithelial cancer. As used herein, the term “non-epithelial cancer” refers to an invasive malignant tumor derived from non-epithelial tissue that can metastasize to other areas of the body.

The cancer can be a metastatic cancer or a non-metastatic (e.g., localized) cancer. As used herein, the term “metastatic cancer” refers to a cancer in which cells of the cancer have metastasized, e.g., the cancer is characterized by metastasis of a cancer cells. The metastasis can be regional metastasis or distant metastasis, as described herein. In a preferred embodiment, the cancer is a metastatic cancer.

In accordance with one or more embodiments of the present invention, it will be understood that the types of abnormal tissue include, for example, lymph nodes. In accordance with one or more embodiments of the present invention, the method can identify a the sentinel lymph node. The sentinel lymph node is the first lymph node that comes out of the tumor. This has enormous ramifications for breast cancer and melanoma patients. The inventors found that when patients were administered, for example, ICG, this tracer would drain and identify the first draining lymph node. This effect cannot be identified in the usual mouse models, and can only be seen in humans.

In a preferred embodiment, at the time of the imaging, the ICG not only identifies the abnormal nodule, it also locates the first draining lymph node, i.e., the sentinel lymph node.

### Methods of Monitoring Treatment Response

In yet another embodiment, the provided methods may be used for tumor therapy response monitoring. In a preferred embodiment, the invention provides a method of monitoring response to a tumor therapy comprising (a) administering to a patient prior to said tumor therapy the fluorophore preparation; (b) providing said tumor therapy; (c) providing the fluorophore preparation after the tumor therapy; and (d) assessing difference in accumulation of the fluorophore preparation from step (a) and step (c), wherein a greater accumulation of the fluorophore preparation in step (a) versus lesser accumulation in step (c) indicates a positive response to the treatment and/or an effective treatment methodology.

In one embodiment, the pharmaceutical composition is administered before surgical resection of a tumor. Complete surgical removal of tumor tissue is often complicated by invasion of the tumor tissue into surrounding tissues and indefinite margins of the mass. Optionally, surgical resection of a tumor is performed after completion of a therapeutic treatment period. Surgical resection of a tumor can be performed at any time after completion of the therapeutic period, so long as the patient is allowed sufficient time to recover from the administration of the pharmaceutical composition, ionizing radiation, and/or chemotherapy. Desirably, surgical resection of a tumor is performed at least 1 week after completion of the therapeutic period. Preferably, surgical resection of a tumor is performed about 3-15 weeks (e.g., about 4 weeks, 5 weeks, 6 weeks, 7 weeks, 8 weeks, 9 weeks, 10 weeks, 11, weeks, 12 weeks, 13 weeks, or 14 weeks) after completion of the therapeutic period. More preferably, surgical resection of a tumor is performed about 3-6 weeks (e.g., about 4 weeks or 5 weeks) or about 4-10 weeks (e.g., about 6 weeks, 7 weeks, or 8 weeks) after completion of the therapeutic period.

Adjuvant radiation and/or chemotherapy can be administered at any time following surgical resection of the tumor, so long as the patient is allowed sufficient recovery time after surgery. In one embodiment, adjuvant chemotherapy is administered to the patient at least 1 week following surgical resection of the tumor. Preferably, adjuvant chemotherapy is administered about 1 week to about 10 weeks (e.g., about 3 weeks, about 5 weeks, or about 7 weeks) following surgical resection of a tumor, more preferably about 2 weeks to about 4 weeks (e.g., about 3 weeks) following surgical resection of a tumor. Any one or combination of chemotherapeutics can be administered to the patient in any suitable dose as part of adjuvant chemotherapy following surgical resection of a tumor. In one embodiment, adjuvant chemotherapy comprises administration of 5-FU and the folic acid derivative leucovorin to the patient.

### Imaging System

An imaging system useful in the practice of this invention typically includes three basic components: (1) an appropriate energy light source for imaging moiety excitation, (2) a means for separating or distinguishing emissions from energy source used for imaging moiety excitation, and (3) a detection system. This system could be hand-held or incorporated into other useful imaging devices such as surgical goggles, endoscopy, open imaging system, closed imaging system or intraoperative microscopes.

Components of the imaging system of the present invention are those that can be generally used in the optical field, the electronic material field, the medical field, the display device/display field, the optical communication field, the information communication field, and the like.

The “light source” may be a light source that can emit MR excitation light at 600 to 1000 nm, for example, at least about 650 nm, and particularly preferably about 780 nm for excitation of the marker and specifically of the fluorescent material. Examples of light source that can be used include: a variety of laser light sources (e.g., ion lasers, dye lasers, and semiconductor lasers); a variety of lamps such as high-pressure mercury lamps, low-pressure mercury lamps, ultrahigh-pressure mercury lamps, metal halide lamps, halogen lamps, nitrogen lamps, and xenon lamps; and a variety of LEDs. If necessary, the light source may have a different optical filter in order to achieve the optimal excitation wavelength.

The detector can be a charge-coupled device CCD, complementary metal-oxide semiconductor (CMOS), spectrometer or avalanche photodiode (APD). An APD can detect weak optical signals due to the internal gain in the detector itself. Because the APD acts as a passively quenched circuit, when it detects single photons an electric field is generated that is sufficiently high to sustain the flow of an avalanche current. Other approaches that rely on external electronic amplification of a weak signal introduce a high background. Additional advantages of the APD include a high quantum efficiency and time resolution, which, if necessary, would allow us to temporally gate the detection and separate cell autofluorescence from probe fluorescence. Because the APDs can count single photons of light, they have the sensitivity to detect single cancer cells that have activated Prosense 750 or any other molecular probe. Indeed, others have created a solid-state microarray detector with APDs that can detect single molecules.

The term “photographing means” refers to a means for creating fluorescence image data that constitute an observation image by detecting NIR fluorescence at, for example, about 600 to 1000 nm, at least about 650 nm, and about 780 nm, emitted by the excited fluorescent material. A means having such functions can be adequately used. Examples of such photographing means include CCD cameras and CMOS cameras. Image data may be created as still image data or moving image data. The photographing means may comprise different types of optical filters for selectively detecting NIR fluorescence at about for example, about 600 to 1000 nm, at least about 650 nm, and about 780 nm. In addition, the photographing means may comprise a surgical laparoscope.

The term “image displaying means” refers to a means for displaying image data output from a photographing means in the form of an observation image. Examples of such image displaying means include CRT displays, liquid crystal displays, organic EL displays, plasma displays, and projection displays. A person who carries out the present invention can obtain a desired observation image by adequately adjusting the amount of light in a preferable manner while viewing an observation image displayed by an image displaying means.

In addition, the imaging system of the present invention can further comprise a means generally used in the field of fluorescence imaging such as a recording means for recording image data photographed by a photographing means, a reflection board for irradiating a subject with excitation light from a light source, and a laser scanner.

The imaging system may have a large depth of field making it less sensitive to small vibrations or motions made by larger macro-like motions of the handheld instrument. An image stabilization subsystem could be employed. Inertial sensor (gryo and accelerometer) would be placed on the hand held portion to detect motion and provide a compensation. The compensation could be moving the image sensor, or lens or employing digital image enhancement.

Further, the present invention relates to a method for detecting a lesion in a living body using the above imaging system. The method comprises the following steps of:

(a) positioning a marker comprising a fluorescent material at the site of a lesion and/or in the vicinity of a lesion in a living body;

(b) irradiating the marker with NIR excitation light from a light source from outside a living body or an organ or tissue of a living body; and

(c) detecting NIR fluorescence emitted from the excited fluorescent material. The imaging device may have, for example, two components: an energy source (ie. excitation) and a capture device (i.e., camera, detector):
1. The energy source should be at a wavelength that can excite the ICG fluorophore. ICG absorbs mainly between 600 nm and 900 nm, though typically energy sources above 750 nm are used. The energy source can be from any source of illumination or electric powered light source such as electron-stimulated, incandescent (ie. halogen), electroluminescent (ie. LED), gas discharge (ie. xenon), laser (ie. laser diode). If a higher energy source is used, the energy from the light source has the potential to kill the tissues retaining the ICG.
2. Any detector or capture device (ie. digital, video camera, CCD) that can collect near-infrared energy will be able to detect the fluorescence from the abnormal nodule. Indocyanine green emits between 750 nm and 950 nm. Additional lighting to provide white-light illumination is also feasible and will not interfere with the fluorescent imaging. A combination of lights and filters can be used to create the impression of a glowing tumor are feasible. Devices can also be added to capture spectroscopic data from the tissue being interrogated. Devices to convert the near-infrared signal to a visible signal are useful.

The imaging device can be mounted over the patient, hand-held device, attached to a long lens system (ie. minimally invasive cameras, telescopes, endoscope, esophagoscope, colonoscope, laparoscope, thoracoscope long lens, capsule endoscope). The imaging device can also be ingested or implanted in the patient. It can capture signal through alternative detectors, however, it does requires excitation energy at the correct frequency for ICG.

This approach does visualize tumors up to 2 cm in depth without amplification. In order to enhance the depth of penetration into the tissue in order to obtain images deeper into tissues, the capture device can record scatter information from the signal that is being emitted from the excited ICG fluorophore in the tissue. This can enhance the depth of penetration of the capture device. Examples of such technology include optical coherence tomography.

This approach does visualize abnormal tissues that can be in the range of the resolution of the human eye. The imaging system can improve the resolution by multiple zoom approaches including capturing spectroscopic signal at the single cell level. It can also use standard amplification devices such as zoom lenses.

The invention will be illustrated in more detail with reference to the following Examples, but it should be understood that the present invention is not deemed to be limited thereto.

### EXAMPLES

### Example 1

### NIR Labeling of Small Animals In Vivo

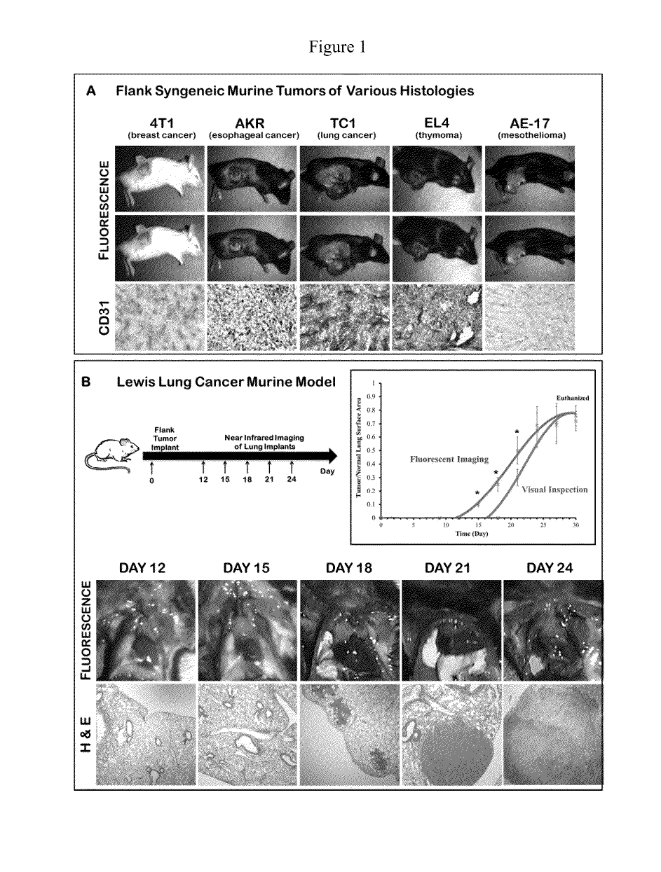

Initially, in order to determine if the EPR effect could deliver a MR contrast agent to solid tumors, we conducted a proof-of-concept study in multiple animal models of malignant disease. Fifty female C57bl/6 mice were injected with five different syngeneic cancer cell lines (4T1 breast cancer, TC1 lung cancer, EL4 thymoma, AE17 mesothelioma, AKR esophageal cancer) into their flanks. After the tumors were well established (˜200 mm³), 7.5 mg/kg of ICG was administered via the tail vein. The next day, a tissue spectrometer was used to semi-quantitate fluorescence from the tumor, surrounding tissue and 12 organs. (Mohs 2010) The mean fluorescence from the tumor was 54,238 arbitrary units (au) (range 46,283-60,000). The mean fluorescence from surrounding normal tissues and organs averaged 4863±1254 au. Tumors were harvested, sectioned and assayed for microvessel density to determine if there was a correlation between tumor vascularity and fluorescence (FIG. 1a). As shown in prior studies, tumor labeling with ICG was highly successful in murine models (Madajewski 2012, Kosaka 2011) but did not directly correlate with tumor vascularity.

We then postulated that NIR labeling of tumors may also detect metastatic cancer cells in the lung. C57bl/6 mice (n=65) were injected into the flank (Day 0) with a murine cancer cell line, Lewis Lung Cancer (LLC), which spontaneously metastasizes to the lung. Starting on Day 12, mice (n=9) were injected with 7.5 mg/kg of ICG via tail vein every three days. Flank tumors were imaged as before, and they were found to have a mean and standard deviation tumor fluorescence of 53,290±2668 au. Subsequently, the chest was opened and inspected for NIR emission from pulmonary metastases. We found that imaging of the murine lungs could detect NIR signal from pulmonary metastases as early as Day 15. These deposits were not visible to the un-assisted eye and were as small as 0.4 mm (FIG. 1b) by histology. The mean tumor fluorescence in early small deposits under 1 mm was 39,923±4577 au, well above the background fluorescence (mean 4290 au). Metastatic pulmonary nodules became visible to the un-assisted eye by Day 24 in all mice. This data confirmed our hypothesis that NIR fluorescence could highlight early deposits of tumor cells in normal tissues. It also suggested that the EPR effect is applicable to lung tumors.

### Patient Study Design

Together, these data supported our hypothesis that NIR labeling with systemic ICG is broadly effective for a range of tumors and can detect primary and metastatic tumors in vivo. Based on these preliminary findings, we initiated a pilot study in patients who presented to a surgical clinic with any tumor in their thoracic cavity (lung, pleural space, mediastinum, chest wall). Between January and June 2012, 27 consecutive patients who were candidates for surgical removal of chest and breast tumors were enrolled in this study (Table 1). Patients were given a single peripheral vein injection of 5 mg/kg ICG, 24 hours prior to surgery. All patients agreed to tissue, blood and urine collection as approved by our institutional review board. Their ages ranged from 31 to 78 years (median=65 years). Two surgeons reached a consensus about the clinical stage and operative approach prior to surgery. All enrolled patients were thought to have limited disease, amenable to surgery, and had no metastases (ie. potentially curable). Ten patients had a biopsy before surgery. Preoperative computed axial and positron emission tomography and/or magnetic resonance imaging predicted no metastatic disease in each case. The median tumor size was 2.0 cm (range 0.6-13.0 cm) on preoperative imaging. Each patient had serum obtained before and 24 hours after ICG injection. Fluorometric interrogation of the patients' serum showed that no patient had detectable ICG in their plasma after 20 hours which was consistent with the known half-life of ICG of 3 to 4 minutes.

### Example 2

### Tumor Fluorescence During Surgical Resection

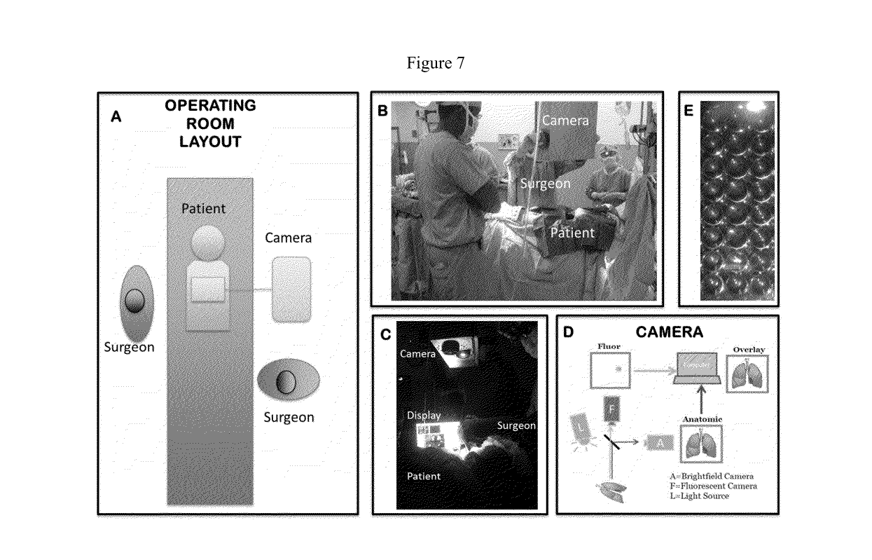

To determine if indocyanine green would be delivered to human tumors, patients undergoing cancer surgery were first imaged in vivo at the onset of the operation. At the time of surgery, the chest was opened and inspected by standard visualization and manual palpation. In all cases, the surgeon could immediately see or feel the tumor. A dual camera head with a brightfield and a NIR output was then used to visualize tumor fluorescence (FIG. 7). In 16 out of 27 (59%) cases, the dual camera head could detect tumor fluorescence at various depths of penetration into the tissue. In the remaining 11 cases, the tumor was located too deep in the organs to image by NIR. The deepest tumor that could be detected was ˜1 cm from the surface of the organ. Attempts to quantitate tumor fluorescence in vivo were not feasible for several reasons including variations in operating room conditions, lack of miniaturized tissue spectrometer with safe laser light source and the inability to control for changes in distance from the specimen to the tip of the spectrometer. However, the subjective impression obtained from visualizing the tumor fluorescence was more than adequate to identify abnormal tissue from normal tissue. All quantitative measurements were made later ex vivo once the specimen had been removed. To examine in vivo data on the distribution of ICG, a complete visual examination of the each patients' skin, muscles (lattismus dorsi, serratus anterior, intercostal, diaphragm), pericardium and heart, aorta, normal lung, lymph nodes, adipose tissues, nerves (phrenic, intercostal), and thymus was performed whenever possible. In all cases, there was no tissue fluorescence except the abnormal tumor.

### Example 3

### Ex Vivo Analysis of Tumor Fluorescence

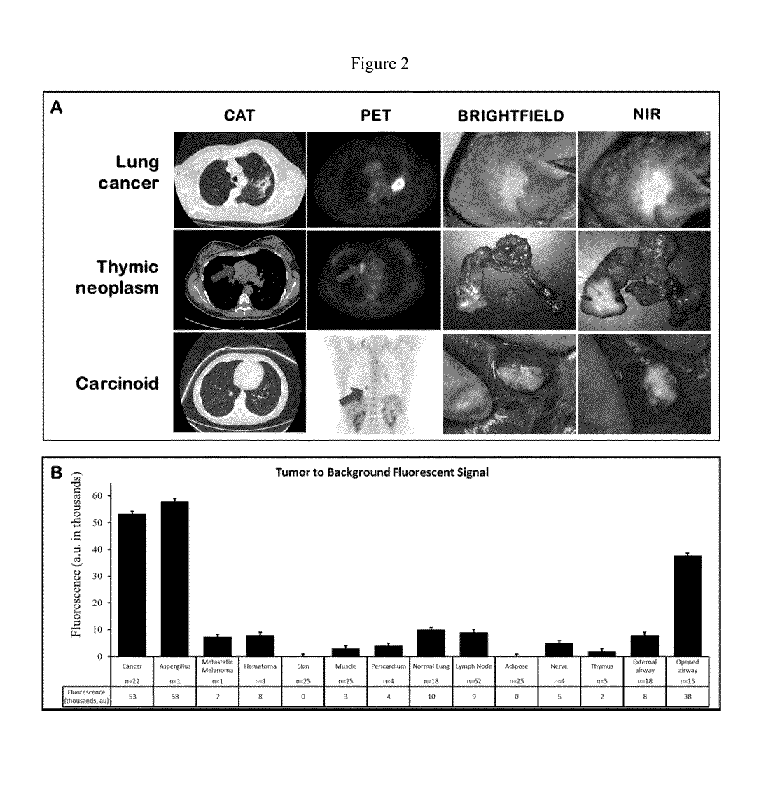

Following in vivo imaging, the patients then underwent a standard-of-care surgical resection of the tumor. Once removed from the patient, the specimen was examined, opened, biopsied and analyzed ex vivo (FIG. 2a). Every case was photo-documented both by brightfield imaging and NIR imaging. Qualitatively, NIR imaging revealed strong fluorescence in 25 out of 27 (92%) masses. Then, the hand held spectrometer was used to semi-quantitate tissue fluorescence. Each tumor had 4 measurements at four perpendicular locations and the center of the tumor (total of 20 measurements/tumor). Mean fluorescence in the human tumors was 53,304±4193 au in 25 out of 27 masses (93%) (FIG. 2b). We conjectured that the center of the tumor might be less fluorescent than the periphery due to necrosis or, conversely, that the center of the tumor might be more fluorescent than the periphery due to increased ICG retention. We found neither to be true. The fluorescence from the tumors was remarkably homogeneous throughout the tumor. We believe this reflects “bleed over” of the fluorescent signal from different areas of the tumor surface. The quality of the image was subjectively better ex vivo than in vivo due to the lack of respiratory motion, light artifact and glare from surrounding tissue retractors. Depending on the different tissues removed from each patient as part of the standard operation, a thorough spectroscopic examination was performed. The average signal diminished from over 50,000 au at the tumor margin to less than 12,000 au within 2 mm of the gross tumor margin. There were some higher signals in areas of atelectatic lung (range 0 to 24,832 au), however, this could easily be distinguished from the tumor by manual palpation. The background signal was measured from the patients' skin, muscles (latissmus dorsi, serratus anterior, intercostal), pericardium, normal lung, lymph nodes, adipose tissues, nerves (intercostal), airway and thymus whenever safely possible (FIG. 2b). There was no evidence of background signal in any surrounding tissue in the body cavity, thus the signal-to-noise ratio was negligible.

Histologically, these tumor biopsies revealed 11 different histological subtypes: 9 pulmonary adenocarcinomas, 5 pulmonary squamous cell carcinomas, 2 invasive ductal carcinomas, 2 melanomas, 2 sarcomas, 1 carcinoid, 1 thymoma, 1 thymic squamous cell carcinoma, 1 adenosquamous carcinoma, 1 MALT lymphoma, 1 aspergilloma and a pulmomary infarct (Table 1). There were 25 cancers and 2 non-cancers (aspergilloma and pulmonary infarct). The two masses that were not fluorescent were a metastatic melanoma (7,342±411 au) and a pulmonary infarct (hematoma, 8,002±554 au). The aspergillus ball, a localized fungus infection, was fluorescent (58,209±1,302 au) likely due to the strong inflammatory reaction surrounding it. Histologically, this aspergillus infection was found to have heavy neutrophilic infiltration, distorted architecture and necrotic exudates. This finding did not detract from the clinical utility of this approach. The pulmonary infarct or hematoma was an old clot secondary to trauma in a 31 year old woman that had been mistaken for a cancer on preoperative imaging. Interestingly, although one melanoma was highly fluorescent (mean 57,210 au), another melanoma in a different patient was minimally fluorescent (mean 7,332 au).

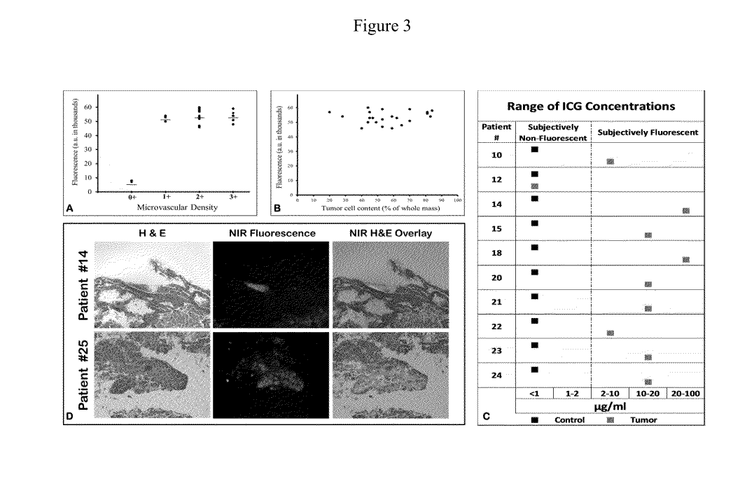

Tumor vascularity is believed to be one of the determinants of adequate delivery of nanoparticles in the EPR effect. To examine this, we compared the fluorescence of the tumor to the microvessel density (MVD) (FIG. 3a). The MVD was designated as 0 (n=2), 1+ (n=6), 2+ (n=11), or 3+ (n=6) based on anti-CD31 antibody expression (two independent investigators). There was no correlation between vascularity and tumor fluorescence. In practical terms, this finding suggests that even minimal vascular tumors have sufficient capacity to accumulate ICG over 24 hours.

We also postulated that the density of cancer cells may correlate with tumor fluorescence. The tumor microenvironment is known to be heterogeneous in cancer cell density ranging from 20-80% of total tumor cells. The remaining cells in the tumor microenvironment are typically a combination of stromal cells and/or infiltrating immune cells. We considered the possibility that tumors with higher cancer cell density would be more likely to retain ICG. In our series, the average cancer cell content in the tumor was 56% (range 20-84%). Again, there was no correlation between the tumor cell density and fluorescence (FIG. 3b).

To quantify the concentration of ICG in the resected tissues, normal (control) and cancerous tissues were imaged ex vivo alongside a standard control panel of known concentrations of ICG aliquots mixed with plasma from the same patient. Our system was able to detect concentrations between 0 μg/ml and 2000 μg/ml. Control tissues of normal lung incidentally removed with each specimen were also analyzed using the same methodology and showed undetectable concentrations of ICG. However, in the tumors, there was a broad range of ICG concentration. In 9 of the 10 specimens, the ICG concentration ranged from 2 to 100 μg/ml (FIG. 3c). No tumor fluoresced above an ICG concentration of 100 μg/ml. However, from a subjective point-of-view, the surgeon was not able to identify any tumors as more or less fluorescent. One cancer (metastatic melanoma) had less than 1 μg/ml of ICG and was not subjectively fluorescent to the surgeon. We also attempted to measure ICG concentration in the tissue by homogenizing punch biopsies and measuring fluorescence in a standard desk fluorometer, however, this approach was unreliable.

In order to determine the location of ICG accumulation within the tumor masses, biopsies were examined by NIR fluorescence microscopy. These studies revealed a consistently heterogeneous pattern of ICG deposition within the tumors. ICG was not confined to the extracellular space as fluorescent overlay images indicated ICG signal coming from individual cells rather than the tumor interstitium or necrotic areas (FIG. 3d). This mosaic of fluorescent signals on a microscopic level gave a uniform appearance of a fluorescent tumor on a macroscopic view (FIG. 2a).

### Example 4

### In Situ Identification of Residual Tumor Deposits after Tumor Resection

Next, we sought to determine the clinical value of tumor fluorescence for cancer patients by examining two circumstances that a surgeon would benefit from this technology: the search for metastases and the verification of disease-free resection margins.

### Identification of Metastases

Thus, prior to closing the body cavity but after the tumor was removed, the chest or breast was inspected visually and by manual palpation for sites of cancer metastases. In all cases, the two surgeons agreed there were no metastatic lesions before imaging. Then, to validate the surgeons' clinical decision, the chest was imaged for NIR fluorescence. In 2 out of 25 cancer cases (8%), the imaging system detected fluorescence in sites greater than 5 cm distant from the primary tumor. Immediate frozen biopsy and intraoperative consultation by a pathologist confirmed that these sites contained metastatic cancer: these lesions harbored metastatic adenocarcinoma and metastatic osteosarcoma cells, respectively.

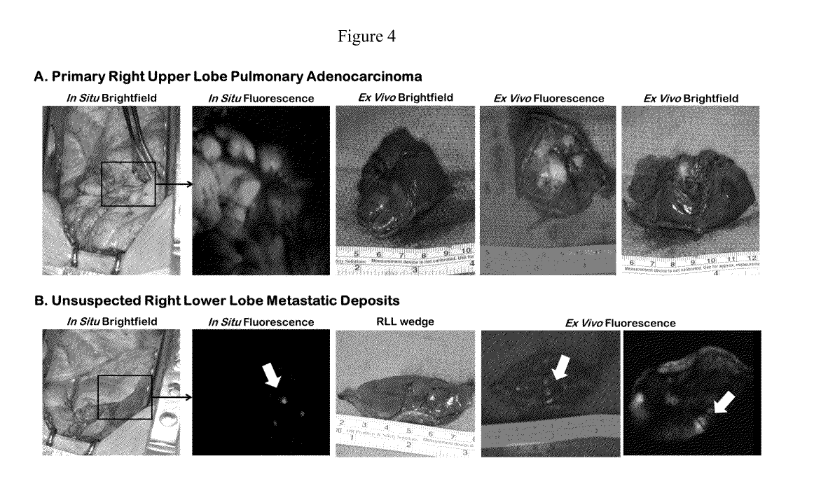

As an example, Patient #2 was a 65 year old male with a clinical diagnosis of a Stage IA right upper lobe lung cancer (FIG. 4a). After removing his primary tumor, the surgeons did not feel or visualize any metastatic lesions. However, upon imaging the chest, there were three sites in the right lower pulmonary lobe that had bright fluorescence (>48,000 au) (FIG. 4b). An excisional biopsy was performed, and imaging again confirmed the presence of small, non-palpable tumor deposits in the specimen. Rapid frozen section and review by a pathologist confirmed metastatic adenocarcinoma. The smallest metastatic nodule detected was 0.4 mm in diameter. A mediastinal lymph node dissection did not reveal metastatic disease. If this patient had undergone a right upper lobectomy based purely on the surgeons' assessment without imaging, he would have been designated to have stage IA lung cancer. The metastatic nodules would not have been discovered because of their location in a different lobe. In addition, he would not have received postoperative chemotherapy because there was no evidence of disease in his lymph nodes. As a result, he would have likely recurred in the future. This delay in diagnosis may have permitted the metastatic nodules to grow and become refractory to standard chemotherapy.

However, since this patient was discovered to have stage IV lung cancer at the time of the operation, he was started on postoperative chemotherapy within 2 weeks following the surgery. Despite being deemed to have stage 1V lung cancer, this patient is still alive at one year without recurrence. By starting therapy while the lesions were still relatively small and radiographically invisible, it is possible that the chemotherapy controlled the minimal tumor burden before it progressed and became refractory to adjuvant therapies.¹⁷ Thus, intraoperative imaging improved clinical staging and likely improved his outcome.

### Identification of Retained Disease at Surgical Margins

The other application of tumor fluorescence during surgery was to confirm that resection was complete and that no tumor cells were left behind in the surgical field. Thus, once the surgeons had completed each case and decided they had obtained disease clearance at the surgical site, the surgical wound and resection field was imaged.

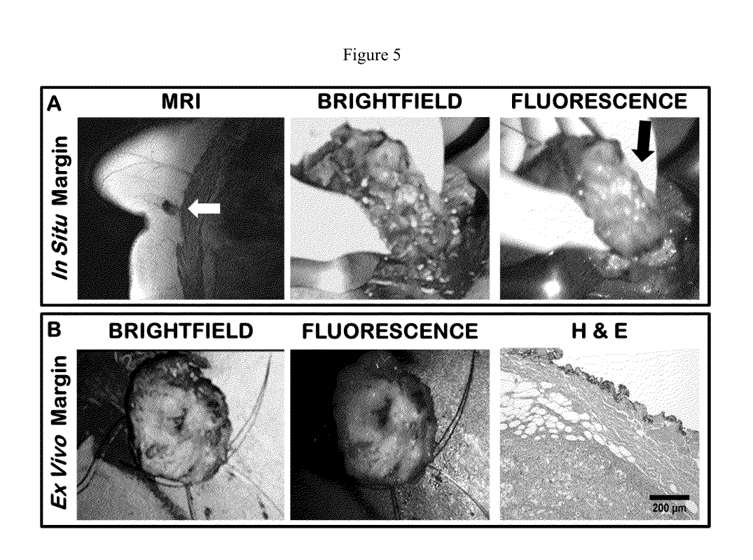

As an example, Patient #24 was a 55 year old female that presented with a 1.7 cm biopsy-proven right breast infiltrative ductal carcinoma. She was consented for breast conserving surgery (a partial lumpectomy and sentinel lymph node biopsy). In the operating room, the mass was markedly fluorescent (mean 52,748 au). As the tumor was resected, strong fluorescence was seen from the posterior margin of the tumor (FIG. 5a). However, by palpation and visual inspection, it appeared to be disease-free to the surgeon (FIG. 5b). As a precaution, a new posterior margin was resected. This new margin did not appear fluorescent. On pathological review, invasive ductal carcinoma was less than 1 mm from the posterior margin in the original surgical specimen (FIG. 5b). The new posterior margin was free of tumor. Thus, intraoperative tumor fluorescence helped improve disease clearance at the margin, and potentially spared the patient a second operation. This patient ultimately received postoperative radiation therapy and is currently disease-free.

In another case, the surgeons felt the surgical margin was too close to the tumor and believed cancer cells had been retained. Therefore, in order to assist with this intraoperative decision to remove more tissue, the imaging system was used to examine the resection margin for fluorescence. The imaging system determined the margin to contain no tumor cells. A pathologist reviewed the specimen and confirmed there no cancer cells at the margin. Therefore, the tumor fluorescence provided a useful adjunct to help refute a surgeons' subjective opinion about retained cancer cells. This patient is disease-free at 7 months.

### Summary of Clinical Outcomes

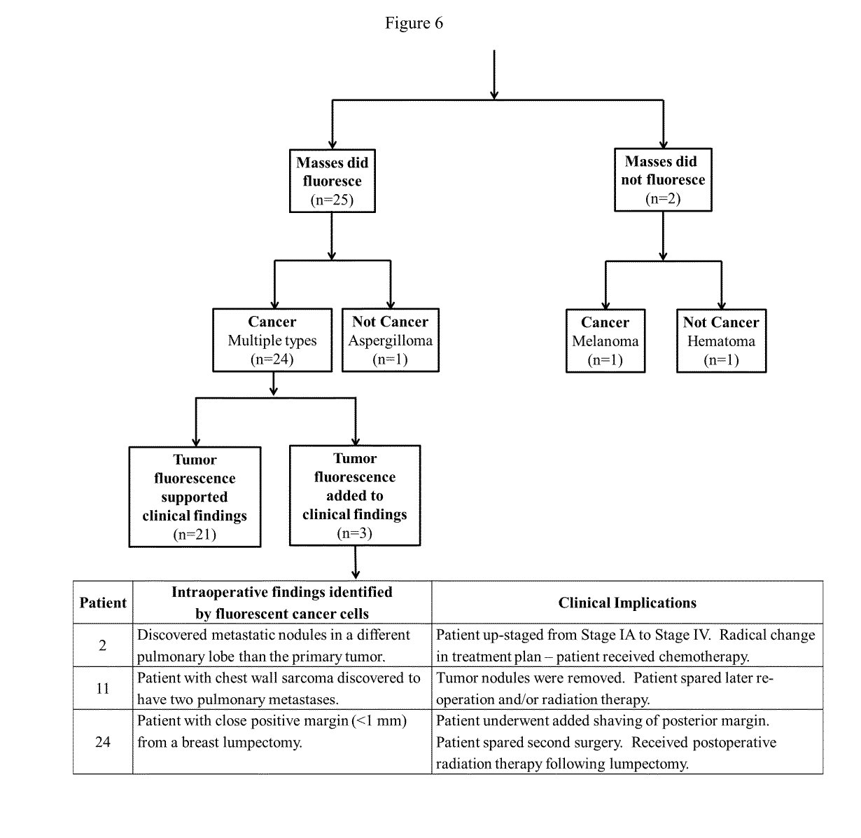

In summary, in this pilot study, no patients were lost to follow up, and there were no obvious toxicities, adverse events or deaths related to injection of ICG. All patients were eligible for the analysis and were alive at the submission of this manuscript. Overall, we imaged 11 different tumor histological subtypes in 27 patients. We found that 2 out of 27 patients (7.4%) were upstaged by intraoperative imaging of fluorescent tumors (FIG. 6). Another patient was spared a re-operation because she was discovered to have a near-positive margin on a breast lumpectomy. Finally, in one other patient, intraoperative imaging allayed the suspicions of the surgeons and spared the patient a larger resection.

### Example 5

### Cell Lines

The murine esophageal carcinoma cell line, AKR, was derived from mouse esophageal squamous epithelia with cyclin D1 over expression via Epstein-Ban virus ED-L2 promoter in p53 deficient genetic backgrounds. (Predina 2011). The murine lung cancer cell line, TC1, was derived from mouse lung epithelial cells immortalized with HPV-16 E6 and E7 and transformed with the c-Ha-ras oncogene.²¹ The metastatic NSCLC cell line, murine Lewis lung carcinoma (LLC), was obtained from American Type Culture Collection (ATCC) (Manassas, Va.). AE17 is an asbestos-derived murine mesothelioma cell line. EL4 was obtained from ATCC and is derived from a mouse lymphoma induced by 9,10-dimethyl-1,2-benzanthracene exposure. 4T1 also obtained from ATCC, is a metastatic murine mammary tumor line that is 6-thioguanine resistant.

Except for TC1 and AE17, cell lines were cultured and maintained in high-glucose DMEM (Dulbecco's Modified Eagle's Medium, Mediatech, Washington D.C.) supplemented with 10% fetal bovine serum (FBS; Georgia Biotechnology, Atlanta, Ga.), 1% penicillin/streptomycin, and 1% glutamine. TC1 and AE17 cell lines were cultured in RPMI (RPMI 1640 Medium, Mediatech, Washington D.C.) 10% FBS, 1% penicillin/streptomycin, and 1% glutamine. Cell lines were regularly tested and maintained negative for Mycoplasma spp.

### Example 6

### Murine Studies

Female C57BL/6 (B6, Thy1.2), BALB/c, athymic Ncr-nu/nu and B6-12931 hybrid mice mice were purchased from Charles River Laboratories and Jackson Laboratories. All mice were maintained in pathogen-free conditions and used for experiments at ages 8 week or older. The Animal Care and Use Committees of the Children's Hospital of Philadelphia and the University of Pennsylvania approved all protocols in compliance with the Guide for the Care and Use of Laboratory Animals. Tumor cells for subcutaneous injections were suspended in 100 μL PBS. Tumor volume was calculated using the formula (π×long-axis×short-axis²)/6.

Surgery was performed on mice bearing flank tumors using an established partial resection model.²² Surgery was performed when tumors reached ˜200 mm3. Mice were anesthetized with intramuscular ketamine (80 mg/kg) and xylazine (10 mg/kg), shaved, and the surgical field sterilized prior to surgery. Initially the mice were imaged to detect NIR signal and then subsequently a 1 to 2 cm incision was made adjacent to the tumor and the tumor was exposed using standard blunt dissection technique. After imaging, the incision was closed using sterile silk 4-0 sutures. Buprenorphine (0.2 mg/kg) was administered at the time of surgery and 6 hours postoperatively to provide analgesia. Preoperative treatment was unknown to the investigator performing surgery and making tumor measurements.

### Example 7

### Human Studies to Assess Primary Tumor, Tumor Margins and Surgical Margins

All human studies were approved by the University of Pennsylvania Institutional Review Board. All patients underwent informed consent. All patients understood additional tissue may require resection based on findings from intraoperative imaging, though the magnitude of the operation would not be significantly altered. The ethical aspects of removing cancer cells if they were found despite the nature of a clinical study were discussed in detail with the institutional review boards. The sequence of the operation was as follows. First, the surgeons performed the standard incision and inspected the body cavity for the nodule using their hands and eyes, dimmed the operating room lights, and then the nodule was examined for fluorescence. The nodule was photo-documented both by white light and fluorescence. Next, the planned cancer resection was performed. Following removal of the tumor, the specimen was again examined ex vivo for tumor fluorescence before sending it to pathology. Finally, the lights were dimmed for a second time, and the open body cavity was inspected for residual fluorescent cancer cells in tumor deposits anywhere in the operative site and at the margins of the resection. Frozen section biopsies were performed when indicated. All specimens were sent for permanent histopathology.

### Reagents

Pharmaceutical grade indocyanine green (ICG) was purchased from Akorn Pharmaceuticals (Lake Forest, Ill.). Mice received 7.5 mg/kg ICG via tail vein 20 hours prior to surgery. Dogs received 5 mg/kg ICG intravenous 24 hours before surgery. Human patients received 5 mg/kg ICG intravenous 18-32 hours prior to surgery.

### Immunohistochemistry

Tissues were harvested and bisected with one half either placed in Tissue-Tek OCT and stored at −80° C. or in formalin for paraffin sectioning. To detect endothelial cells, monoclonal CD31 (mAB390) was raised from hybridoma supernatant and purified. Frozen tumor sections were prepared as previously described.²³ CD31 expression was quantified by counting the number of positively staining cells in four high-powered (×400) fields.²⁴

### Fluorescence Microscopy

Tumor biopsies in each case were taken in the operating room and immediately frozen in optimal cutting temperature compound to −20 degrees. Biopsy were then cut into 20μ thick sections and mounted with a gylcerine-based mounting media. The samples were then examined using an Olympus® IX51 fluorescent microscope equipped with an indocyanine green specific filter set (Chroma® 49030). Image capture was achieved using a PixeLink® NIR CCD camera (PL-B741 EU). Each sample was then subsequently stained with hematoxylin and eosin and re-imaged using white light. Fluorescent images were further processed using ImageJ® to give green pseudo-color to fluorescent signal and then these images were subsequently overlaid to create color-NIR images.

### Flow Cytometric Analysis of Tumors

Flow cytometry was performed as previously described.¹⁷ Briefly, tumors were minced into fine pieces in digestion buffer containing 0.1 mg/mL DNase 1 and 2.0 mg/mL collagenase type IV (Sigma, St. Louis, Mo.). Samples were incubated in digestion buffer at 37° C. for 30 minutes, filtered through a 70-μm filter, and washed twice with R10. After preparation, cells were incubated for 30 minutes at 4° C. with appropriate antibodies CD45 and EPCAM (BD Biosciences, San Diego, Calif. and eBiosciences, New Jersey). Flow cytometry was completed using a Becton Dickinson FACS Calibur flow cytometer (San Jose, Calif.), and analyzed using FlowJo software (Ashland, Oreg.).

### Near Infrared Fluorescent Imaging System

As schematically depicted in FIG. 7, our intraoperative device is a single integrated dual camera imaging system with a multi-line solid-state light source to provide both excitation light of the fluorescent probe and white light illumination. Specific filters are selected to split the fluorescent labeled cancer cells to a specific camera. The CCD cameras are aligned and secured to a metal plate such that an overlay of two images allows for precise location of the fluorescent probe within the tissue. The signals are processed by a computer and are co-displayed and overlaid on a color monitor. During a surgical operation, the 780 nm and optical channels provide information about tumor presence or absence (as judged by contrast agent accumulation). In the final display, the tumor overlay is translucent, so that the surgeon can still see anatomical detail through the overlaid region. A boom stand (BioMediCon®) was used to place the imaging device above the patient during surgery.

### Quantification of Tissue Fluorescent Intensity

A hand-held near infrared spectrometer has been previously described in detail.¹⁸ In brief, a Raman probe detector was incorporated into a cylindrical stainless steel sampling head integrated with a 5 m, two-fiber cable; one for laser excitation and the other for light collection. The sampling head and fiber cable were coupled via an FC connector to a spectrometer. The combined sampling head and spectrometer system has a wavelength range of 800-930 nm with 0.6 nm spectral resolution for near infrared (NIR) fluorescence measurement. The excitation light was provided by a 785 nm, 100 mW continuous-wave diode laser. The signal can be semi-quantitated from 0 to 60,000 arbitrary units (au).

### Semi-Quantification of ICG Concentration in Plasma

Techniques have been previously described to use fluorometry to quantify concentrations of ICG in plasma. We obtained aliquots of each patient's blood before injection with ICG. These samples were spun down at 1100 RPM for 10 minutes and aliquots of plasma were removed. 2 mg of ICG was dissolved in 1 ml of plasma and serially diluted thirteen times to a concentration of 125 μg per liter. 100 microliters of each of these dilutions were pipetted into a separate wells in a standard black 96 well plate along with one well of 100 microliters of plasma without ICG. At the time of operation, each patient had their plasma obtained and serially diluted six times to a final ratio of 1:1000. Plates were examined using a SpetraMax Fluorometer® with a peak excitation of 805 nm and 830 nm. Each well was scanned 6 times. Readings were export as Microsoft Excel® spread sheets. No measurable ICG was detected in the patient's plasma 24 hours after ICG injection.

### Semi-Quantification of ICG Concentration in Tumors

To quantify the maximum concentration of ICG in each tumor, 10 patients had their plasma collected at the time of operation. Two mg of ICG was dissolved in 1 ml of plasma and serially diluted 8 times to a final ratio of 1:20,000. 100 μL of stock solution and each dilution was then placed in consecutive wells in a black 96 well plate. When tumors were imaged ex-vivo, this plate was concurrently imaged. These images were then imported into ImageJ® software. Region of interest (ROI) data was taken from each of the 9 wells and from the tumor. The maximum fluorescent signal from the tumor was compared to the wells to determine the highest concentration per tumor. Each tumor was then categorized as have an ICG concentration between given the standards in the known panel.

TABLE 1
Clinical findings from intraoperative fluorescence

| | | | | Tumor Fluorescence | | | |
|---|---|---|---|---|---|---|---|
| | | | | Identified | Identified | Identified | |
| | | Size | | primary | metastatic | positive | |
| Case | Histology | (cm) | Site^{†} | tumor | disease | margins | Clinical Details |
| 1 | Adenocarcinoma | 1.2 | LLL | + | | | |
| 2 | Adenocarcinoma | 3.6 | RUL | + | + | | Patient discovered to have multiple metastatic nodules |
| | | | | | | | in a different pulmonary lobe. Patient up-staged from |
| | | | | | | | Stage IA (5-yr survival 75%) to Stage IV (5-yr |
| | | | | | | | survival 2%). Radical change in treatment plan. |
| 3 | Squamous Cell | 2.6 | LLL | + | | | |
| 4 | Carcinoid | 1.7 | RLL | + | | | |
| 5 | Adenocarcinoma | 1.2 | LUL | + | | | |
| 6 | MALT | 1.6 | LUL | + | | | |
| | Lymphoma | | | | | | |
| 7 | Adenocarcinoma | 0.6 | LUL | + | | − | Intraoperative evaluation of suspected positive margin |
| | | | | | | | was incorrect by surgeon but correct by tumor |
| | | | | | | | fluorescence. Important because patient was unable to |
| | | | | | | | tolerate a larger operation. |
| 8 | Pulmonary | 1.9 | RML | | | | Benign lesion did not glow. |
| | Infarct | | | | | | |
| 9 | Adenosquamous | 3.6 | RUL | + | | | |
| 10 | Melanoma | 1.5 | LUL | + | | | |
| 11 | Osteosarcoma | 3 | LUL | + | + | | Patient with chest wall sarcoma was found to have two |
| | | | | | | | un-suspected metastases. Tumor nodules were |
| | | | | | | | removed. Patient spared later re-operation and/or |
| | | | | | | | radiation. |
| 12 | Melanoma | 2.3 | RLL | | | | Metastatic melanoma did not glow. Note: Tumor was |
| | | | | | | | dark black color. |
| 13 | Thymoma | 3.1 | Thymus | + | | | |
| 14 | Adenocarcinoma | 2 | RML | + | | | |
| 15 | Squamous cell | 2.6 | RUL | + | | | |
| 16 | Aspergilloma | 5.5 | LUL | + | | | |
| 17 | Squamous Cell | 7 | Thymus | + | | | |
| 18 | Squamous Cell | 2.6 | RUL | + | | | |
| 19 | Liposarcoma | 13 | Chest | + | | | |
| | | | Wall | | | | |
| 20 | Adenocarcinoma | 11 | LUL | + | | | |
| 21 | Squamous Cell | 3 | LUL | + | | | |
| 22 | Adenocarcinoma | 1.5 | LUL | + | | | |
| 23 | Squamous Cell | 1.9 | LLL | + | | | |
| 24 | Ductal | 1.7 | RB | + | | + | Patient felt to have a positive margin at the time of |
| | Adenocarcinoma | | | | | | surgery due to tumor fluorescence at posterior margin. |
| | | | | | | | Pathology confirmed margin <1 mm, new posterior |
| | | | | | | | margin was disease free. |
| 25 | Adenocarcinoma | 1.6 | LUL | + | | | |
| 26 | Adenocarcinoma | 0.8 | RUL | + | | | |
| 27 | Ductal | 0.5 | RB | + | | | |
| | Adenocarcinoma | | | | | | |

^{†}Abbreviations: LUL (left upper pulmonary lobe), RUL (right upper pulmonary lobe), RML (right middle pulmonary lobe), LLL (left lower pulmonary lobe), RLL (right lower pulmonary lobe), RB (right breast)

While the invention has been described in detail and with reference to specific examples thereof, it will be apparent to one skilled in the art that various changes and modifications can be made therein without departing from the spirit and scope thereof.

What is claimed is:

1. A method for identifying abnormal tissue in a subject during an operative, radiologic or endoscopic procedure, said method comprising:
(a) administering to the subject a fluorophore preparation comprising an effective amount of at least one fluorophore, wherein said at least one fluorophore comprises indocyanine green (ICG) in a total systemic dose of at least about 2 mg/kg of body weight of the subject, wherein the administration is systemic;
(b) conducting said procedure after a waiting period subsequent to said administration, wherein said waiting period is selected from the group consisting of at least about 12 hours, about 24 hours, about 36 hours, about 48 hours, between about 12 to about 24 hours, between about 24 to about 36 hours, between about 36 to about 48 hours;
(c) during the procedure, illuminating the area of interest with an illumination source emitting electromagnetic radiation (emr) having at least one wavelength which interacts with ICG dye, the emr having a wavelength of from about 600 nm to about 1000 nm;
(d) imaging the abnormal tissue with an imaging device, wherein the abnormal tissue displays significantly more fluorescence caused by the fluorophore preparation;
(e) optionally imaging the lymph nodes draining from the abnormal tissue;
(f) optionally, treating sites of abnormal tissue by external beam radiation, laser therapy, or surgical removal.

2. A method in accordance with claim 1, wherein said procedure is an operative procedure, radiologic or an endoscopic procedure.

3. A method in accordance with claim 1, wherein said procedure is an endoscopic procedure.

4. The method of claim 1, wherein the preparation is administered intravenously.

5. The method of claim 1, wherein the fluorophore preparation comprises ICG administered in a total systemic dose of about 2 to 10 mg/kg of body weight of the subject.

6. The method of claim 1, wherein the fluorophore preparation comprises ICG administered in a total systemic dose of at least about 2 to about 3 mg/kg of body weight of the subject.

7. A method in accordance with claim 1, wherein the fluorophore preparation further comprises a fluorophore selected from the group consisting of cyanine dyes, streptocyanines dyes, hemicyanine dyes, closed chain cyanine dyes, methylene blue (MB), IR-786, CW800-CA, and combinations thereof.

8. The method of claim 1, wherein the abnormal tissue is selected from the group consisting of a neoplasia, a tumor, a metastasis, a lymph node, a sentinel lymph node, draining lymph node and combinations thereof.

9. The method of claim 8, wherein the abnormal tissue is a neoplasia selected from the group consisting of breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal gangllioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, lymphomas, malignant melanomas, epidermoid carcinomas, lymph node, sentinel lymph node, and combinations thereof.

10. The method of claim 9, wherein the abnormal tissue is pancreatic cancer, breast cancer, or colon cancer.

11. A method in accordance with claim 1, wherein said procedure further comprises treating sites of abnormal tissue by external beam radiation, laser therapy, and/or surgical removal.

12. A method in accordance with claim 1, wherein said illumination source is selected from the group consisting of electron-stimulated, incandescent, halogen, electroluminescent, LED, gas discharge, xenon, laser, and laser diode.

13. A method in accordance with claim 1, wherein said illumination source emits emr having at least one wavelength which interacts with ICG dye, the emr having a wavelength of at least 650 nm

14. A method in accordance with claim 1, wherein said illumination source emits emr having at least one wavelength which interacts with ICG dye, the emr having a wavelength of about 780 nm.

15. A method in accordance with claim 1, wherein said imaging device is selected from the group consisting of spectrometer, digital, video camera, and CCD.

16. A method in accordance with claim 1, wherein a combination of lights and filters is used to create the impression of a glowing abnormal tissue.

17. A method in accordance with claim 1, further comprising imaging devices capable of capturing spectroscopic data from the tissue being imaged.

18. A method in accordance with claim 1, further comprising imaging devices to convert the near-infrared signal to a visible signal.

19. A method in accordance with claim 1, wherein the imaging device is selected from the group consisting of devices which can be mounted over the patient, hand-held devices, devices which are attached to a long lens system, minimally invasive cameras, telescopes, endoscopes, esophagoscopes, colonoscopes, laparoscopes, thoracoscope long lens, capsule endoscopes, and combinations thereof.

20. A method in accordance with claim 1, wherein the imaging device is ingested or implanted in the subject.

21. A method in accordance with claim 1, wherein the imaging device can record scatter information from the signal that is being emitted from the excited fluorophore preparation in the abnormal tissue in order to improve the depth of penetration and imaging quality.

22. A method in accordance with claim 1, wherein the imaging device comprises an optical coherence tomography device.

23. A method in accordance with claim 1, wherein the imaging device is modified to excite different fluorophores separately and simultaneously capture the emission from the different fluorophores, further wherein computer software then represents this data simultaneously for an observer.

24. A kit comprising a vial containing a sterile preparation of a fluorophore preparation for systemic administration comprising an effective amount of at least one fluorophore, wherein said at least one fluorophore comprises indocyanin green (ICG), and instructions for use, wherein said instructions direct administration of ICG at a total systemic dose of at least about 2 to 5 mg/kg of body weight of the subject, but up to 10 mg/kg, and direct a waiting period after administration of the fluorophore preparation is selected from the group consisting of about 12 hours, about 24 hours, about 36 hours, about 48 hours, between about 12 to about 24 hours, between about 24 to about 36 hours, between about 36 to about 48 hours.

25. The kit of claim 24, wherein the fluorophore preparation further comprises a fluorophore selected from the group consisting of methylene blue (MB), IR-786, CW800-CA, and combinations thereof.

26. A method for identifying abnormal tissue in a subject during an operative or endoscopic procedure, said method comprising:
(a) administering to the subject a fluorophore preparation comprising an effective amount of at least one fluorophore, wherein said at least one fluorophore comprises indocyanin green (ICG), wherein the administration is systemic, further wherein the ICG is administered in a total systemic dose of about 2 to 10 mg/kg of body weight of the subject;
(b) conducting said procedure after a waiting period subsequent to said administration, wherein said waiting period is at least about 12 hours;
(c) during the procedure, illuminating the area of interest with an illumination source emitting electromagnetic radiation (emr) having at least one wavelength which interacts with ICG dye, the emr having a wavelength of from about 600 nm to about 1000 nm;
(d) imaging the abnormal tissue, optionally with an imaging device, wherein the abnormal tissue displays significantly more fluorescence caused by the fluorophore preparation;
(e) optionally imaging the lymph nodes draining from the abnormal tissue;
(f) optionally, treating sites of abnormal tissue by external beam radiation, laser therapy, or surgical removal.

27. A method for identifying abnormal tissue in a subject during an operative or endoscopic procedure, said method comprising:
(a) administering to the subject a fluorophore preparation comprising an effective amount of at least one fluorophore, wherein said at least one fluorophore comprises indocyanine green (ICG), wherein the administration is systemic, further wherein the ICG is administered in a total systemic dose of at least about 2 to about 5 mg/kg of body weight of the subject, but up to 10 mg/kg;
(b) conducting said procedure after a waiting period subsequent to said administration, wherein said waiting period is at least about 24 hours;
(c) during the procedure, illuminating the area of interest with an illumination source emitting electromagnetic radiation (emr) having at least one wavelength which interacts with ICG dye, the emr having a wavelength of from about 600 nm to about 1000 nm;
(d) imaging the abnormal tissue, optionally with an imaging device, wherein the abnormal tissue displays significantly more fluorescence caused by the fluorophore preparation;
(e) optionally imaging the lymph nodes draining from the abnormal tissue;
(f) optionally, treating sites of abnormal tissue by external beam radiation, laser therapy, or surgical removal.
